SEQUENCE LISTING (44 sequences)

SEQ ID NO: 1 - protein, 124 aa, synthetic construct
  source 1..124: note = Dupilumab HCVR
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVS

SEQ ID NO: 2 - protein, 112 aa, synthetic construct [sequence omitted]

SEQ ID NO: 3 - protein, 8 aa, synthetic construct
  source 1..8: note = Dupilumab HCDR1
GFTFRDYA

SEQ ID NO: 4 - protein, 8 aa, synthetic construct
  source 1..8: note = Dupilumab HCDR2
ISGSGGNT

SEQ ID NO: 5 - protein, 18 aa, synthetic construct
  source 1..18: note = Dupilumab HCDR3
AKDRLSITIRPRYYGLDV

SEQ ID NO: 6 - protein, 11 aa, synthetic construct
  source 1..11: note = Dupilumab LCDR1
QSLLYSIGYNY

SEQ ID NO: 7 - skipped

SEQ ID NO: 8 - protein, 9 aa, synthetic construct
  source 1..9: note = Dupilumab LCDR3
MQALQTPYT

SEQ ID NO: 9 - protein, 451 aa, synthetic construct
  source 1..451: note = Dupilumab HC
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 10 - protein, 219 aa, synthetic construct
  source 1..219: note = Dupilumab LC
DIVMTQSPLSLPVTPGEPASISCRSSQSLLYSIGYNYLDWYLQKSGQSPQLLIYLGSNRASGVPDRFSGSGSGTDFTLKISRVEAEDVGFYYCMQALQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - DNA, 375 bp, synthetic construct
  source 1..375: mol_type = other DNA
gaggtgcagctggtggagtctgggggaggcttggtccagcctggggggtccctgagactctcctgtgcagcctctggattcacctttagtaacttttggatgacctgggtccgccaggctccagggaaggggctggagtgggtggccaacatgaaccaagatggaagtgagaaatactatgtggactctgtgaagggccgattcaccatctccagagacaacgccaagagctcactgtatctgcaaatgaacagcctgagagccgaggacacggctgtgtattactgtgcgagagatcgggaatattgtattagtaccagctgctatgatgactttgactactggggccagggaaccctggtcaccgtctcctca

SEQ ID NO: 12 - protein, 125 aa, synthetic construct
EVQLVESGGGLVQPGGSLRLSCAASGFTFSNFWMTWVRQAPGKGLEWVANMNQDGSEKYYVDSVKGRFTISRDNAKSSLYLQMNSLRAEDTAVYYCARDREYCISTSCYDDFDYWGQGTLVTVSS

SEQ ID NO: 13 - DNA, 24 bp, synthetic construct
  source 1..24: mol_type = other DNA
ggattcacctttagtaacttttgg

SEQ ID NO: 14 - protein, 8 aa, synthetic construct
GFTFSNFW

SEQ ID NO: 15 - DNA, 24 bp, synthetic construct
  source 1..24: mol_type = other DNA
atgaaccaagatggaagtgagaaa

SEQ ID NO: 16 - protein, 8 aa, synthetic construct
MNQDGSEK

SEQ ID NO: 17 - DNA, 54 bp, synthetic construct
  source 1..54: mol_type = other DNA
gcgagagatcgggaatattgtattagtaccagctgctatgatgactttgactac

SEQ ID NO: 18 - protein, 18 aa, synthetic construct
ARDREYCISTSCYDDFDY

SEQ ID NO: 19 - DNA, 324 bp, synthetic construct
  source 1..324: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaa

SEQ ID NO: 20 - protein, 108 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 21 - DNA, 18 bp, synthetic construct
  source 1..18: mol_type = other DNA
cagagcattagcagctat

SEQ ID NO: 22 - protein, 6 aa, synthetic construct
QSISSY

SEQ ID NO: 23-24 - skipped (2 entries)

SEQ ID NO: 25 - DNA, 30 bp, synthetic construct
  source 1..30: mol_type = other DNA
caacagagttacagtacccctccgatcacc

SEQ ID NO: 26 - protein, 10 aa, synthetic construct
QQSYSTPPIT

SEQ ID NO: 27 - DNA, 372 bp, synthetic construct
  source 1..372: mol_type = other DNA
gaagtacagcttgtagaatccggcggaggactggtacaacctggaagaagtcttagactgagttgcgcagctagtgggtttacattcgacgattacagcatgcattgggtgaggcaagctcctggtaaaggattggaatgggttagcgggatatcatggaactcaggaagcaagggatacgccgacagcgtgaaaggccgatttacaatatctagggacaacgcaaaaaactctctctaccttcaaatgaactctcttagggcagaagacacagcattgtattattgcgcaaaatacggcagtggttatggcaagttttatcattatggactggacgtgtggggacaagggacaacagtgacagtgagtagc

SEQ ID NO: 28 - protein, 124 aa, synthetic construct
EVQLVESGGGLVQPGRSLRLSCAASGFTFDDYSMHWVRQAPGKGLEWVSGISWNSGSKGYADSVKGRFTISRDNAKNSLYLQMNSLRAEDTALYYCAKYGSGYGKFYHYGLDVWGQGTTVTVSS

SEQ ID NO: 29 - DNA, 24 bp, synthetic construct
  source 1..24: mol_type = other DNA
gggtttacattcgacgattacagc

SEQ ID NO: 30 - protein, 8 aa, synthetic construct
GFTFDDYS

SEQ ID NO: 31 - DNA, 24 bp, synthetic construct
  source 1..24: mol_type = other DNA
atatcatggaactcaggaagcaag

SEQ ID NO: 32 - protein, 8 aa, synthetic construct
ISWNSGSK

SEQ ID NO: 33 - DNA, 51 bp, synthetic construct
  source 1..51: mol_type = other DNA
gcaaaatacggcagtggttatggcaagttttatcattatggactggacgtg

SEQ ID NO: 34 - protein, 17 aa, synthetic construct
AKYGSGYGKFYHYGLDV

SEQ ID NO: 35 - DNA, 372 bp, synthetic construct
  source 1..372: mol_type = other DNA
gaagtacagcttgtagaatccggcggaggactggtacaacctggaagaagtcttagactgagttgcgcagctagtgggtttacattcgacgattacagcatgcattgggtgaggcaagctcctggtaaaggattggaatgggttagcgggatatcatggaactcaggaagcatcggatacgccgacagcgtgaaaggccgatttacaatatctagggacaacgcaaaaaactctctctaccttcaaatgaactctcttagggcagaagacacagcattgtattattgcgcaaaatacggcagtggttatggcaagttttattattatggaatggacgtgtggggacaagggacaacagtgacagtgagtagc

SEQ ID NO: 36 - protein, 124 aa, synthetic construct
EVQLVESGGGLVQPGRSLRLSCAASGFTFDDYSMHWVRQAPGKGLEWVSGISWNSGSIGYADSVKGRFTISRDNAKNSLYLQMNSLRAEDTALYYCAKYGSGYGKFYYYGMDVWGQGTTVTVSS

SEQ ID NO: 37 - DNA, 24 bp, synthetic construct
  source 1..24: mol_type = other DNA
gggtttacattcgacgattacagc

SEQ ID NO: 38 - protein, 8 aa, synthetic construct
GFTFDDYS

SEQ ID NO: 39 - DNA, 24 bp, synthetic construct
  source 1..24: mol_type = other DNA
atatcatggaactcaggaagcatc

SEQ ID NO: 40 - protein, 8 aa, synthetic construct
ISWNSGSI

SEQ ID NO: 41 - DNA, 51 bp, synthetic construct
  source 1..51: mol_type = other DNA
gcaaaatacggcagtggttatggcaagttttattattatggaatggacgtg

SEQ ID NO: 42 - protein, 17 aa, synthetic construct
AKYGSGYGKFYYYGMDV

SEQ ID NO: 43 - protein, 117 aa, synthetic construct
  source 1..117: note = R1103 HCVR
EVQLQQSGPELVKPGASVRMSCKASGYTFTDYNIHWVKQSHGKSLEWIGYIYPNNGDNGYNQKFRGKATLTVDKSSSTAYMELRSLTSDDSAVYYCARGRLRYFDVWGTGTTVTVSS

SEQ ID NO: 44 - protein, 111 aa, synthetic construct
  source 1..111: note = R1103 LCVR
NIVLTQSPASLAVSLGQRATISCRASESVDNYGHSFMHWYQQKPGQPPKLLIYLASNLESGVPARFSGSGSRTDFTLTLDPVEADDAATYYCQQYNEDPPTFGSGTKLEIK

(19) United States
(12) Patent Application Publication
(10) Pub. No.: US 2024/0350625 A1
(43) Pub. Date: Oct. 24, 2024

(54) COMBINATION OF IL-4/IL-13 PATHWAY INHIBITORS AND PLASMA CELL ABALATION FOR TREATING ALLERGY

(71) Applicant: REGENERON PHARMACEUTICALS, INC., Tarrytown, NY (US)

(72) Inventors: Seblewongel ASRAT, New York, NY (US); Andre LIMNANDER, New York, NY (US); Jamie M. ORENGO, Cortlandt Manor, NY (US); Andrew J. MURPHY, Croton-on-Hudson, NY (US); George D. YANCOPOULOS, Yorktown Heights, NY (US)

(21) Appl. No.: 18/610,188

(22) Filed: Mar. 19, 2024

Related U.S. Application Data
(62) Division of application No. 16/825,955, filed on Mar. 20, 2020, now Pat. No. 11,964,016.
(60) Provisional application No. 62/822,022, filed on Mar. 21, 2019.
(60) Provisional application No. 62/957,550, filed on Jan. 6, 2020.

(51) Int. Cl. A61K 39/395; A61K 39/00; A61P 37/08

(52) U.S. Cl. CPC A61K 39/3955; A61P 37/08; A61K 2039/507

(57) ABSTRACT

The present disclosure provides methods for treating allergy comprising selecting a patient with an allergy and administering a therapeutically effective amount of an IL-4/IL-13 pathway inhibitor (e.g., an anti-IL-4 receptor antibody or antigen-binding fragment thereof) in combination with a therapeutically effective amount of an agent that depletes plasma cells (e.g., an anti-BCMA/anti-CD3 bispecific antibody). In certain embodiments, a plasma cell ablating agent such as an anti-BCMA/anti-CD3 bispecific antibody ablates the plasma cells, including IgE+ plasma cells, while the IL-4/IL-13 pathway inhibitor prevents the generation of new IgE+ plasma cells, thus eliminating allergen-specific IgE in the patient.

### CROSS-REFERENCE TO RELATED APPLICATIONS

This application claims priority to U.S. Provisional Patent Application Nos. 62/822,022, filed Mar. 21, 2019, and 62/957,550, filed Jan. 6, 2020, the entire content of each of which is incorporated by reference herein.

### SEQUENCE STATEMENT

The instant application contains a Sequence Listing, which has been submitted electronically in ASCII format and is hereby incorporated by reference in its entirety. Said ASCII copy, created on Mar. 20, 2020, is named SequenceList_10581USU1.TXT and is 22 kilobytes in size.

### FIELD OF THE INVENTION

The present disclosure relates to methods for treating allergy comprising administering to a subject in need thereof a therapeutically effective amount of an IL-4/IL-13 pathway inhibitor in combination with a therapeutically effective amount of a plasma cell ablating agent.

### BACKGROUND

Allergies and allergic diseases are serious medical conditions with consequences ranging from non-life-threatening responses that resolve over time to life-threatening effects such as anaphylaxis. Allergic reactions can result from contact or exposure to a variety of products such as certain food items, insect venom, plant-derived material (e.g., pollen), chemicals, drugs/medications, and animal dander. The pathophysiology of allergy is influenced by a complex interplay between Immunoglobulin E (IgE)-mediated sensitization, the immune system, and environmental factors. Current treatment options for allergies include avoidance, pharmacological symptom treatment and prophylaxis using allergen-specific immunotherapies (SIT). Unfortunately, these current treatment strategies are often inadequate, costly, impractical or involve significant risk. For example, avoidance of allergen is not always possible and can negatively impact on patient and caregiver quality of life. Immunotherapeutic approaches, on the other hand, involve deliberate administration of allergen to susceptible individuals and are therefore inherently risky with the potential for unwanted severe allergic reactions or anaphylaxis. Accordingly, an unmet need exists in the art for novel therapeutic approaches that prevent or treat allergies or allergic responses and reduce the risk of developing an allergic response.

### SUMMARY

In one aspect, the present disclosure provides methods for treating an allergy, allergic reaction, or allergic disorder, for preventing or reducing the severity of an allergic reaction, or for reducing or eliminating allergen-specific serum IgE in a subject. In some embodiments, the method comprises administering to a subject (e.g., a subject having an allergy, an allergic disorder, a mast cell activation disorder, or mastocytosis) an IL-4/IL-13 pathway inhibitor and a plasma cell ablating agent.

In certain embodiments, the present disclosure includes methods of treating allergy or preventing or reducing the severity of an allergic reaction, comprising: (a) selecting a subject with an allergic disease or disorder, a mast cell activation disorder or mastocytosis; and (b) administering to the subject in need thereof a therapeutically effective amount of an IL-4/IL-13 pathway inhibitor and a therapeutically effective amount of a plasma cell ablating agent.

In certain embodiments, the present disclosure includes methods of treating allergy or preventing or reducing the severity of an allergic reaction, comprising: (a) selecting a subject with an allergic disease or disorder, a mast cell activation disorder, or mastocytosis; and (b) administering to the subject in need thereof a therapeutically effective amount of an IL-4/IL-13 pathway inhibitor and a plasma cell ablating agent.

In certain embodiments, the present disclosure includes methods for treating allergy or preventing or reducing the severity of an allergic reaction, comprising: (a) selecting a subject with an allergic disease or disorder, a mast cell activation disorder, or mastocytosis, wherein the subject is on a background therapy regimen comprising one or more doses on an IL-4/IL-13 pathway inhibitor; and (b) administering at least one dose of a plasma cell ablating agent.

In one embodiment, the administration of the IL-4/IL-13 pathway inhibitor prevents generation of new IgE+ plasma cells and the administration of the plasma cell ablating agent leads to elimination of bone marrow-resident IgE+ plasma cells, thus eliminating allergen-specific serum IgE.

In certain embodiments, the present disclosure includes methods for increasing the efficacy and/or tolerability of an immunotherapy regimen in a subject having an allergy. In some embodiments, the method comprises administering to the subject an IL-4/IL-13 pathway inhibitor and a plasma cell ablating agent prior to or concurrent with the immunotherapy regimen. In some embodiments, the immunotherapy regimen is an oral immunotherapy (OIT) regimen. In some embodiments, the immunotherapy regimen is a subcutaneous immunotherapy (SCIT) regimen. In some embodiments, the immunotherapy is an allergen-specific immunotherapy regimen for a food allergen (e.g., a peanut allergen). In some embodiments, the immunotherapy is an allergen-specific immunotherapy regimen for an environmental allergen.

In one embodiment of the methods disclosed herein, the allergic disease or disorder is selected from the group consisting of allergic asthma, hay fever, chronic urticaria, food allergy, pollen allergy, and allergy due to an environmental allergen. In one embodiment, the subject is at a risk of anaphylaxis due to an allergen. In one embodiment, the subject has seasonal allergy. In one embodiment, the subject has a severe allergy. In one embodiment, the subject has an allergy due to one or more allergens selected from the group consisting of milk, a dairy product, egg, celery, sesame, wheat, meat, fruit, soy, fish, shellfish, a sugar, peanuts, a legume, a tree nut, dust, dust mite, pollen, insect venom, mold, animal fur, animal dander, wool, latex, a metal, a household cleaner, a detergent, medication, cosmetics, perfumes, a drug such as penicillin, sulfonamides, or salicylate, therapeutic monoclonal antibodies (e.g., cetuximab), ragweed, grass and birch. In one embodiment, the allergen is contained in a food item selected from the group consisting of milk, a dairy product, egg, celery, sesame, wheat, meat, fruit, soy, fish, shellfish, a sugar, peanuts, a legume, and a tree nut. In one embodiment, the allergen is a non-food allergen selected from the group consisting of dust, dust mite, pollen, insect venom, mold, animal fur, animal dander, wool, latex, a metal, a household cleaner, a detergent, medication, cosmetics, perfumes, a drug such as penicillin, sulfonamides, or salicylate, therapeutic monoclonal antibodies (e.g., cetuximab), ragweed, grass and birch.

In one embodiment of the methods disclosed herein, the IL-4/IL-13 pathway inhibitor is selected from the group consisting of an anti-IL-4 antibody, an anti-IL-13 antibody, an anti-IL-4/IL-13 bispecific antibody, an IL-4 receptor (IL-4R) inhibitor, an IL-4 trap, an IL-13 trap, and an anti-IL-4R antibody. In one embodiment, the IL-4/IL-13 pathway inhibitor is an anti-IL-4 antibody (e.g., pascolizumab). In another embodiment, the IL-4/IL-13 pathway inhibitor is an anti-IL-13 antibody (e.g., tralokinumab, lebrikizumab, dectrekumab, GSK679586, or MEDI7836). In another embodiment, the IL-4/IL-13 pathway inhibitor is an anti-IL-4/IL-13 bispecific antibody (e.g., romilkimab). In another embodiment, the IL-4/IL-13 pathway inhibitor is an IL-4R inhibitor (e.g., an IL-4 mutein such as pitrakinra or an anti-IL-4R antibody). In another embodiment, the IL-4/IL-13 pathway inhibitor is an anti-IL-4R antibody. In another embodiment, the IL-4/IL-13 pathway inhibitor is an IL-4 trap or IL-13 trap.

In one embodiment, the IL-4/IL-13 pathway inhibitor is an anti-IL-4R antibody or an antigen-binding fragment thereof. In one embodiment, the anti-IL-4R antibody comprises a heavy chain variable region (HCVR) that includes three heavy chain complementarity determining regions (CDRs) (HCDR1, HCDR2 and HCDR3) and a light chain variable region (LCVR) that includes three light chain CDRs (LCDR1, LCDR2 and LCDR3), wherein: HCDR1 has an amino acid sequence of SEQ ID NO: 3; HCDR2 has an amino acid sequence of SEQ ID NO: 4; HCDR3 has an amino acid sequence of SEQ ID NO: 5; LCDR1 has an amino acid sequence of SEQ ID NO: 6; LCDR2 has an amino acid sequence of SEQ ID NO: 7; and LCDR3 has an amino acid sequence of SEQ ID NO: 8. In another embodiment, the anti-IL-4R antibody includes a heavy chain variable region (HCVR) comprising the amino acid sequence of SEQ ID NO: 1 and a light chain variable region (LCVR) comprising the amino acid sequence of SEQ ID NO: 2. In another embodiment, the anti-IL-4R antibody includes a heavy chain and a light chain, wherein the heavy chain has an amino acid sequence of SEQ ID NO: 9. In another embodiment, the anti-IL-4R antibody includes a heavy chain and a light chain, wherein the light chain has an amino acid sequence of SEQ ID NO: 10. In another embodiment, the anti-IL-4R antibody includes a heavy chain and a light chain, wherein the heavy chain has an amino acid sequence of SEQ ID NO: 9 and the light chain has an amino acid sequence of SEQ ID NO: 10. In another embodiment, the IL-4/IL-13 pathway inhibitor is dupilumab or a bioequivalent thereof. In another embodiment, the IL-4/IL-13 pathway inhibitor is selected from the group consisting of dupilumab, pascolizumab, AMG317, MEDI2045, MEDI9314, tralokinumab, lebrikzimab, anrukinzumab, dectrekumab, GSK679586, MEDI7836, romilkimab, an IL-4 trap, an IL-13 trap, AER-003, and pitrakinra.

In one embodiment of the methods disclosed herein, the plasma cell ablating agent is selected from the group consisting of a B-cell maturation antigen (BCMA) targeting agent, a proteasome inhibitor, a histone deacetylase inhibitor, a B-cell activating factor (BAFF) inhibitor, and an inhibitor of A proliferation inducing ligand (APRIL; CD256). In one embodiment, the BCMA targeting agent is selected from the group consisting of an anti-BCMA/anti-CD3 bispecific antibody, a chimeric antigen receptor against BCMA, and an anti-BCMA antibody conjugated to a cytotoxic drug.

In one embodiment, the plasma cell ablating agent is an anti-BCMA/anti-CD3 bispecific antibody or antigen-binding fragment thereof comprising (a) a first antigen-binding domain that specifically binds to BCMA; and (b) a second antigen-binding domain that specifically binds CD3. In one embodiment, the first antigen-binding domain comprises three heavy chain CDRs (HCDR1, HCDR2 and HCDR3) contained with a heavy chain variable region (HCVR) comprising the amino acid sequence of SEQ ID NO: 12; and three light chain CDRs (LCDR1, LCDR2 and LCDR3) contained with a light chain variable region (LCVR) comprising the amino acid sequence of SEQ ID NO: 20. In a further embodiment, HCDR1 has an amino acid sequence of SEQ ID NO: 14; HCDR2 has an amino acid sequence of SEQ ID NO: 16; HCDR3 has an amino acid sequence of SEQ ID NO: 18; LCDR1 has an amino acid sequence of SEQ ID NO: 22; LCDR2 has an amino acid sequence of SEQ ID NO: 24; and LCDR3 has an amino acid sequence of SEQ ID NO: 26. In another embodiment, the second antigen-binding domain comprises three heavy chain CDRs (HCDR1, HCDR2 and HCDR3) contained with a heavy chain variable region (HCVR) comprising the amino acid sequence selected from the group consisting of SEQ ID NOs: 28 and 36; and three light chain CDRs (LCDR1, LCDR2 and LCDR3) contained with a light chain variable region (LCVR) comprising the amino acid sequence of SEQ ID NO: 20. In a further embodiment, HCDR1 has an amino acid sequence of SEQ ID NO: 30 or 38; HCDR2 has an amino acid sequence of SEQ ID NO: 32 or 40; HCDR3 has an amino acid sequence of SEQ ID NO: 34 or 42; LCDR1 has an amino acid sequence of SEQ ID NO: 22; LCDR2 has an amino acid sequence of SEQ ID NO: 24; and LCDR3 has an amino acid sequence of SEQ ID NO: 26. In one embodiment, the anti-BCMA/anti-CD3 bispecific antibody comprises: (a) a first antigen-binding domain that binds specifically to BCMA and that comprises three heavy chain CDRs and three light chain CDRs wherein the six CDRs HCDR1-HCDR2-HCDR3-LCDR1-LCDR2-LCDR3 comprise the amino acid sequences of SEQ ID NOs: 14-16-18-22-24-26; and (b) a second antigen-binding domain that binds specifically to CD3 and that comprises three heavy chain CDRs and three light chain CDRs wherein the six CDRs HCDR1-HCDR2-HCDR3-LCDR1-LCDR2-LCDR3 comprise the amino acid sequences of SEQ ID NOs: 30-32-34-22-24-26. In one embodiment, the anti-BCMA/anti-CD3 bispecific antibody comprises: (a) a first antigen-binding domain that binds specifically to BCMA and that comprises three heavy chain CDRs and three light chain CDRs wherein the six CDRs HCDR1-HCDR2-HCDR3-LCDR1-LCDR2-LCDR3 comprise the amino acid sequences of SEQ ID NOs: 14-16-18-22-24-26; and (b) a second antigen-binding domain that binds specifically to CD3 and that comprises three heavy chain CDRs and three light chain CDRs wherein the six CDRs HCDR1-HCDR2-HCDR3-LCDR1-LCDR2-LCDR3 comprise the amino acid sequences of SEQ ID NOs: 38-40-42-22-24-26. In one embodiment, the anti-BCMA/anti-CD3 bispecific antibody comprises: (a) a first antigen-binding domain comprising a HCVR of SEQ ID NO: 12 and a LCVR of SEQ ID NO: 20; and (b) a second antigen-binding domain comprising a HCVR of SEQ ID NO: 28 and a LCVR of SEQ ID NO: 20. In one embodiment, the anti-BCMA/anti-CD3 bispecific antibody comprises: (a) a first antigen-binding domain comprising a HCVR of SEQ ID NO: 12 and a LCVR of SEQ ID NO: 20; and (b) a second antigen-binding domain comprising a HCVR of SEQ ID NO: 36 and a LCVR of SEQ ID NO: 20.

In one embodiment, the IL-4/IL-13 pathway inhibitor is administered prior to the plasma cell ablating agent. In one embodiment, wherein the IL-4/IL-13 pathway inhibitor is administered after the plasma cell ablating agent. In one embodiment, the administration of the IL-4/IL-13 pathway inhibitor in combination with a plasma cell ablating agent blocks IgE production and eliminates allergen-specific IgE from the serum in the patient as compared to a subject treated with either therapeutic as monotherapy.

In another embodiment, one or more doses of the IL-4/IL-13 pathway inhibitor are administered in combination with one or more doses of the plasma cell ablating agent. In another embodiment, at least one dose of the IL-4/IL-13 pathway inhibitor includes about 0.1 to about 50 mg/kg of the subject's body weight. In another embodiment, at least one dose of the IL-4/IL-13 pathway inhibitor includes about 0.05 to about 600 mg of the inhibitor. In another embodiment, at least one dose of the plasma cell ablating agent includes about 0.1 mg/kg to about 20 mg/kg of the subject's body weight. In another embodiment, at least one dose of the plasma cell ablating agent includes about 0.05 to about 500 mg of the agent.

In another embodiment, the method further includes administering at least one additional therapeutic agent or therapy. In another embodiment, the additional therapeutic agent or therapy includes an IgE antagonist, an anti-histamine, an anti-inflammatory agent, a corticosteroid, a leukotriene antagonist, a mast cell inhibitor, a bronchial dilator, a decongestant, epinephrine, an IL-1 antagonist, an IL-5 antagonist, an IL-31 antagonist, an IL-33 antagonist, an IL-25 antagonist, interferon γ, a TNF antagonist, and/or a TSLP antagonist.

In another aspect, the present disclosure provides pharmaceutical compositions and combinations for treating an allergy, allergic reaction, or allergic disorder, for preventing or reducing the severity of an allergic reaction, for reducing or eliminating allergen-specific serum IgE in a subject, or for increasing the efficacy and/or tolerability of an immunotherapy regimen in a subject having an allergy. In some embodiments, the pharmaceutical composition or combination comprises an IL-4/IL-13 pathway inhibitor (e.g., an anti-IL-4Ra antibody) and a plasma cell ablating agent (e.g., a BCMA targeting agent). In some embodiments, the pharmaceutical composition or combination comprises a therapeutically effective amount of an IL-4/IL-13 pathway inhibitor (e.g., an anti-IL-4Ra antibody) and a therapeutically effective amount of a plasma cell ablating agent (e.g., a BCMA targeting agent). In some embodiments, the pharmaceutical composition or combination comprises a subtherapeutic dose of an IL-4/IL-13 pathway inhibitor (e.g., an anti-IL-4Ra antibody) and/or a plasma cell ablating agent (e.g., a BCMA targeting agent).

In still another aspect, the present disclosure provides for the use of an IL-4/IL-13 pathway inhibitor (e.g., an anti-IL-4Ra antibody) and a plasma cell ablating agent (e.g., a BCMA targeting agent) in the manufacture of a medicament for treating an allergy, allergic reaction, or allergic disorder, for preventing or reducing the severity of an allergic reaction, for reducing or eliminating allergen-specific serum IgE in a subject, or for increasing the efficacy and/or tolerability of an immunotherapy regimen in a subject having an allergy. In some embodiments, one or both of the IL-4/IL-13 pathway inhibitor and a plasma cell ablating agent is used in a therapeutic effective amount. In some embodiments, one or both of the IL-4/IL-13 pathway inhibitor and a plasma cell ablating agent is used at a subtherapeutic dose.

### BRIEF DESCRIPTION OF THE FIGURES

FIG. 1 is a diagrammatic representation of the house dust mite (HDM) exposure and antibody treatment protocol in accordance with the study described in Example 1.

FIG. 2A shows serum IgE levels upon exposure to HDM for 11 weeks followed by 1-week rest in mice treated with either saline, no antibody, an isotype control antibody, REGN5459 (anti-BCMA×anti-CD3 bispecific antibody), REGN1103 (anti-IL-4R antibody), or a combination of REGN5459 and REGN1103 in accordance with the study described in Example 1. The asterisks (*) indicate degree of statistical significance relative to isotype controls (IgGs).

FIG. 2B shows serum IgE levels upon exposure to HDM for 11 weeks followed by 6 weeks rest in mice treated with either saline, no antibody, an isotype control antibody, REGN5459 (anti-BCMA×anti-CD3 bispecific antibody), REGN1103 (anti-IL-4R antibody), or a combination of REGN5459 and REGN1103 in accordance with the study described in Example 1. The asterisks (*) indicate degree of statistical significance relative to isotype controls (IgGs).

FIG. 3 is a diagrammatic representation of the HDM exposure and antibody treatment protocol in accordance with the study described in Example 2.

FIG. 4A shows HDM-specific serum IgE levels in mice subjected to ongoing exposure to HDM and 1 week post-treatment with an anti-BCMA×anti-CD3 bispecific antibody (REGN5459) or an isotype control (REGN4460), in accordance with the study described in Example 2. The asterisks (*) indicate degree of statistical significance relative to isotype controls (IgGs). LLOQ=lower limit of quantification.

FIG. 4B shows HDM-specific serum IgE levels in mice subjected to ongoing exposure to HDM and 3 weeks post-treatment with an anti-BCMA×anti-CD3 bispecific antibody (REGN5459) or an isotype control (REGN4460), in accordance with the study described in Example 2. The asterisks (*) indicate degree of statistical significance relative to isotype controls (IgGs).

FIG. 4C shows HDM-specific serum IgE levels in mice subjected to ongoing exposure to HDM and 5 weeks post-treatment with an anti-BCMA×anti-CD3 bispecific antibody (REGN5459) or an isotype control (REGN4460), in accordance with the study described in Example 2. The asterisks (*) indicate degree of statistical significance relative to isotype controls (IgGs).

FIG. 5 shows the effect of anti-BCMA×anti-CD3 bispecific antibody and anti-IL-4Ra antibody, alone or in combination, on IgE bone marrow plasma cells 5 weeks after administration of anti-BCMA×anti-CD3 bispecific antibody. The asterisks (*) indicate degree of statistical significance relative to isotype controls (IgGs); *p≤0.05; **p≤0.01; ***p≤0.001; ****p<0.0001.

FIG. 6 is a diagrammatic representation of the HDM exposure and antibody treatment protocol in accordance with the study described in Example 4.

FIG. 7 shows the effect of anti-BCMA×anti-CD3 bispecific antibody and anti-IL-4Ra antibody, alone or in combination, on serum HDM-specific IgE levels. The treatment groups are described in Example 4 and Table 7 and are: Group A (saline), Group B (HDM for 12 weeks, no antibody), Group C (HDM for 15 weeks, no antibody), Group D (HDM for 15 weeks, isotype control antibodies), Group E (HDM for 15 weeks, anti-BCMA×anti-CD3 bispecific antibody), Group F (HDM for 15 weeks, anti-IL-4Ra antibody), and Group G (HDM for 15 weeks, anti-IL-4Ra antibody and anti-BCMA×anti-CD3 bispecific antibody). Statistical significance is compared between the treatment groups as shown in the inset. ns=not statistically significant; *p≤0.05; **p≤0.01; ***p≤0.001; ****p<0.0001. LLOQ=lower limit of quantification.

FIGS. 8A-8D show the effect of anti-BCMA×anti-CD3 bispecific antibody, alone or in combination with anti-IL-4Ra antibody, on total bone marrow plasma cells (FIG. 8A), IgE bone marrow plasma cells (FIG. 8B), total splenic plasma cells (FIG. 8C), and IgE splenic plasma cells (FIG. 8D). The treatment groups are described in Example 4 and Table 7 and are: Group A (saline), Group B (HDM for 12 weeks, no antibody), Group C (HDM for 15 weeks, no antibody), Group D (HDM for 15 weeks, isotype control antibodies), Group E (HDM for 15 weeks, anti-BCMA×anti-CD3 bispecific antibody), Group F (HDM for 15 weeks, anti-IL-4Ra antibody), and Group G (HDM for 15 weeks, anti-IL-4Rα antibody and anti-BCMA×anti-CD3 bispecific antibody). Statistical significance is compared between the treatment groups as shown in the inset. ns=not statistically significant; *p≤0.05; **p≤0.01; ***p≤0.001; ****p≤0.0001.

### DETAILED DESCRIPTION

It is to be understood that the present disclosure is not limited to the particular methods and experimental conditions described, as such methods and conditions may vary. It is also to be understood that the terminology used herein is for the purpose of describing particular embodiments only, and is not intended to be limiting, and that the scope of the present disclosure will be limited only by the appended claims.

Unless defined otherwise, all technical and scientific terms used herein have the same meaning as commonly understood by one of ordinary skill in the art to which the disclosure belongs. As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

Although any methods and materials similar or equivalent to those described herein can be used in the practice of the present disclosure, the preferred methods and materials are now described.

### Introduction

Allergic symptoms, including anaphylaxis, are driven by allergen-induced cross-linking of allergen-specific IgE bound to FcεR on effector cells (mast cells and basophils) that can induce mast cell degranulation. In allergic individuals, circulating IgE arises from the antibody-secreting cells in the bone marrow and from B cells that class-switch to produce new IgE-producing cells. The antibody-secreting cells that accumulate in the bone marrow are long-lived and are a source of allergen-specific IgE, even in the absence of the allergen. Further, allergen-specific IgE can be long-lived in an individual, as evidenced by, at least, the following: (1) IgE is maintained in atopic patients in the absence of allergen (Luger et al., Allergol Int 2010, 59:1-8); (2) allergy can be transferred from an atopic patient to a non-atopic individual during a bone marrow transplant from the former to the latter (Garzorz et al., J Eur Acad Dermatol Venereol 2016, 30:1136-1139; Hallstrand et al., Blood 2004, 104:3086-3090); and (3) serum IgE is not abolished in patients with IgE+ B-cell ablation approaches (Gauvreau et al., Sci Transl Med 2014, 6:243ra85).

In initial studies, the inventors of the present disclosure observed that treatment with an anti-IL-4R antibody prevented class-switching and differentiation of B cells to IgE-producing plasma cells in a mouse model of allergen-induced lung inflammation but did not impact the IgE+ plasma cells in the bone marrow during chronic allergen exposure. The inventors thus hypothesized that treatment with an IL-4/IL-13 pathway inhibitor in combination with targeted ablation of the long-lived plasma cells (including IgE+ plasma cells) might lead to blocking IgE production from the newly generated IgE+ plasma cells as well as from IgE+ plasma cells in the bone marrow. As shown herein, this therapeutic combination can significantly reduce or completely block the production of allergen-specific IgE in an animal model of allergen (HDM)-induced type 2 lung inflammation. Such a combination of therapeutic agents could therefore also be useful in treating allergic disease in atopic subjects.

### Methods of Treating Allergy

In one aspect, the present disclosure relates to the surprising results obtained by the inventors, wherein administration of an IL-4/IL-13 pathway inhibitor (e.g., an anti-IL-4R antibody) in combination with a plasma cell ablating agent (e.g., an anti-BCMA/anti-CD3 bispecific antibody) resulted in complete elimination of allergen-specific IgE in the serum of subjects in the chronic allergen-induced lung inflammation model.

Accordingly, in some embodiments the present disclosure provides methods for treating, ameliorating, or reducing the severity of at least one symptom or indication of allergy in a subject. In some embodiments, the present disclosure provides methods for preventing or reducing the severity of an allergic reaction in a subject. In one aspect, the disclosed methods include selecting a subject with an allergic disease or disorder, a mast cell activation disorder, or mastocytosis; and administering to the subject in need thereof a therapeutically effective amount of an IL-4/IL-13 pathway inhibitor (e.g., an anti-IL-4 antibody, an anti-IL-13 antibody, an anti-IL-4/IL-13 bispecific antibody, an IL-4 receptor (IL-4R) inhibitor, an anti-IL-4R antibody, or any other “IL-4/IL-13 pathway inhibitor” as described herein) and a therapeutically effective amount of a plasma cell ablating agent (e.g., an anti-BCMA/anti-CD3 bispecific antibody, or any other “plasma cell ablating agent” as described herein).

In the present disclosure, references to any particular anti-IL-4R antibody and/or any particular plasma cell ablating agent are provided to illustrate a representative IL-4/IL-13 pathway inhibitor and a representative plasma cell ablating agent, respectively, and do not limit the scope of the disclosure as combinations of other IL-4/IL-13 pathway inhibitors and plasma cell ablating agents may also be used.

As used herein, the terms “treat,” “treating,” or the like, mean to alleviate allergic symptoms, eliminate the causation of allergic symptoms either on a temporary or permanent basis, or to prevent or slow the appearance of allergic symptoms in a subject. The terms, as used herein, also include reducing or abrogating allergen-specific serum IgE to prevent an allergic reaction. In some embodiments, the terms refer to decreasing the level of serum allergen-specific IgE by at least 50%, 60%, 70%, 80% or more as compared to baseline, upon administration of an IL-4/IL-13 pathway inhibitor in combination with a plasma cell ablating agent as provided by methods of the present disclosure. In certain embodiments, the terms refer to eliminating the level of serum allergen-specific IgE as compared to baseline, upon administration of an IL-4/IL-13 pathway inhibitor in combination with a plasma cell ablating agent as provided by methods of the present disclosure.

As used herein, the expression “a subject in need thereof” means a human or non-human animal that exhibits one or more symptoms or indicia of allergy or atopy, and/or who has been diagnosed with allergy to an allergen. The terms “subject” and “patient” are used interchangeably herein. In certain embodiments, the term “subject in need thereof” includes subjects that are at an increased risk for developing an allergy or an allergic response to an allergen. In certain embodiments, the term includes subjects that show allergen sensitization to one or more allergens. In certain embodiments, the methods of the present disclosure may be used to treat subjects that show elevated levels of one or more serum biomarkers including, but not limited to, total IgE, allergen-specific IgE, thymus and activation-regulated chemokine (TARC), pulmonary and activation-regulated chemokine (PARC), lactate dehydrogenase (LDH), and/or periostin. For example, in some embodiments the methods of the present disclosure comprise administering an IL-4/IL-13 pathway inhibitor in combination with a plasma cell ablating agent to patients with elevated levels of allergen-specific serum IgE.

The term “subject in need thereof” also includes subjects with an allergic disease or disorder selected from the group consisting of allergic asthma, hay fever, chronic urticaria, food allergy, pollen allergy, and allergy due to an environmental (non-food) allergen. The term also includes subjects that suffer from severe allergy due to one or more allergens. For example, in some embodiments, a subject has a “severe” allergy if the subject exhibits one or more severe symptoms of an allergic response, such as a symptom of anaphylaxis (e.g., difficult/noisy breathing, swelling of tongue, swelling/tightness in throat, difficulty talking and/or hoarse voice, wheeze or persistent cough, nausea/vomiting, persistent dizziness, collapse, or loss of consciousness).

In certain embodiments, the term “subject in need thereof” includes a subject that is susceptible to an allergic reaction or is at an increased risk for developing an allergic reaction to an allergen. For example, the term includes subjects that are at risk of anaphylaxis due an allergen such as peanut or penicillin. In certain embodiments, a subject may be at an increased risk of developing an allergy or an allergic response to an allergen due to sensitization to said allergen. For example, the term includes subjects that show increased levels of serum IgE specific to one or more allergens (“allergen sensitization”), e.g., to one or more food allergens and/or environmental allergens. In some embodiments, the subject has an allergen-specific IgE level of at least about 0.35 kU/L (e.g., for one or more allergens as disclosed herein, such as a food allergen or an environmental allergen, or an allergen selected from the group consisting of milk, a dairy product, egg, celery, sesame, wheat, meat, fruit, soy, fish, shellfish, a sugar, peanuts, a legume, a tree nut, dust, dust mite, pollen, insect venom, mold, animal fur, animal dander, wool, latex, a metal, a household cleaner, a detergent, medication, cosmetics, perfumes, a drug such as penicillin, sulfonamides, or salicylate, therapeutic monoclonal antibodies, ragweed, grass, and birch). In the context of the present disclosure, the term “subject in need thereof” also includes subjects having an atopic disease and subjects that have a disease or disorder selected from the group consisting of atopic dermatitis, asthma, allergic rhinitis, eosinophilic esophagitis and food allergy. The term “subject” also includes subjects with elevated levels of serum total and allergen-specific IgE, or serum chemokines (e.g., CCL17 or CCL27) that may have an increased risk of developing an allergic response. In one aspect, the present disclosure provides methods to decrease the risk of developing allergy or allergic response in susceptible subjects.

As used herein, the terms “allergic response,” “allergic reaction,” “allergic symptom,” and the like, include one or more signs or symptoms selected from the group consisting of urticaria (e.g., hives), angioedema, rhinitis, asthma, vomiting, sneezing, runny nose, sinus inflammation, watery eyes, wheezing, bronchospasm, reduced peak expiratory flow (PEF), gastrointestinal distress, flushing, swollen lips, swollen tongue, reduced blood pressure, anaphylaxis, and organ dysfunction/failure. An “allergic response,” “allergic reaction,” “allergic symptom,” etc., also includes immunological responses and reactions such as, e.g., increased IgE production and/or increased allergen-specific immunoglobulin production.

The term “allergen,” as used herein, includes any substance, chemical, particle or composition which is capable of stimulating an allergic response in a susceptible individual. Allergens may be contained within or derived from a food item such as, e.g., dairy products (e.g., cow's milk), egg, celery, sesame, wheat, meat, soy, fish, shellfish, sugars (e.g., sugars present on meat such as alpha-galactose), peanuts, other legumes (e.g., beans, peas, soybeans, etc.), and tree nuts; an allergen that is contained within or derived from a food item is referred to herein as a “food allergen.” Alternatively, an allergen may be contained within or derived from a non-food item, e.g., an indoor or outdoor environmental allergen such as, e.g., dust (e.g., containing dust mite), pollen, insect venom (e.g., venom of bees, wasps, mosquitos, fire ants, etc.), mold, animal fur, animal dander, wool, latex, metals (e.g., nickel), household cleaners, detergents, medication, cosmetics (e.g., perfumes, etc.), drugs (e.g., penicillin, sulfonamides, salicylate, etc.), therapeutic monoclonal antibodies (e.g., cetuximab), ragweed, grass and birch. Exemplary pollen allergens include, e.g., tree pollens such as birch pollen, cedar pollen, oak pollen, alder pollen, hornbeam pollen, aesculus pollen, willow pollen, poplar pollen, plantanus pollen, tilia pollen, olea pollen, Ashe juniper pollen, and Alstonia scholaris pollen. Other examples of allergens can be found elsewhere herein.

The present disclosure includes methods for treating allergy, including severe allergy, or preventing or reducing the severity of an allergic reaction, comprising administering to a subject in need thereof a therapeutically effective amount of an IL-4/IL-13 pathway inhibitor (e.g., an anti-IL-4R antibody as described herein) in combination with a therapeutically effective amount of a plasma cell ablating agent (e.g., an anti-BCMA×anti-CD3 bispecific antibody as described herein). In some embodiments, the disclosed methods target an allergic disease or disorder, mast cell activation disorder, or mastocytosis. In one embodiment, the allergic disease or disorder is selected from the group consisting of allergic asthma, hay fever, chronic urticaria, food allergy, pollen allergy, and allergy due to an environmental (non-food) allergen. In some embodiments, the allergic disease is a food allergy, e.g., a peanut allergy. In some embodiments, the allergic disease is a severe food allergy.

According to certain embodiments, the present disclosure includes methods of treating allergy or preventing or reducing the severity of an allergic reaction, the methods comprising: (a) selecting a patient with an allergic disease or disorder, a mast cell activation disorder or mastocytosis, wherein the patient is on a background therapy regimen comprising one or more doses of an IL-4/IL-13 pathway inhibitor; and (b) administering at least one dose of a plasma cell ablating agent. The methods, in this aspect, enhance the therapeutic efficacy of the IL-4/IL-13 pathway inhibitor in reducing the serum levels of allergen-specific IgE. In certain embodiments, the patient is on a therapeutic regimen comprising one or more doses of an IL-4/IL-13 pathway and is administered one or more doses of a plasma cell ablating agent, thereby enhancing the anti-allergic effect of the IL-4/IL-13 pathway inhibitor.

In some embodiments, the disclosed methods include administering a therapeutically effective amount of an IL-4/IL-13 pathway inhibitor and a therapeutically effective amount of a plasma cell ablating agent in combination with an additional therapeutic agent or therapy (e.g., regimen or procedure). The additional therapeutic agent or therapy may be administered for increasing anti-allergic efficacy, for reducing toxic effects of one or more therapies and/or for reducing the dosage of one or more therapies. In various embodiments, the additional therapeutic agent or therapy may include one or more of an IgE antagonist, an anti-histamine, an anti-inflammatory agent, a corticosteroid, a leukotriene antagonist, a mast cell inhibitor, a bronchial dilator, a decongestant, epinephrine, an IL-1 antagonist, an IL-5 antagonist, an IL-31 antagonist, an IL-33 antagonist, an IL-25 antagonist, interferon γ, a TNF antagonist, and a TSLP antagonist.

The additional therapeutic agent may be, e.g., another IL-4R antagonist, an IL-1 antagonist (including, e.g., an IL-1 antagonist as set forth in U.S. Pat. No. 6,927,044), an IL-6 antagonist, an IL-6R antagonist (including, e.g., an anti-IL-6R antibody as set forth in U.S. Pat. No. 7,582,298), an IL-13 antagonist, a tumor necrosis factor (TNF) antagonist, an IL-8 antagonist, an IL-9 antagonist, an IL-17 antagonist, an IL-5 antagonist, an IgE antagonist (e.g., an anti-IgE antibody such as omalizumab), a CD48 antagonist, an IL-31 antagonist (including, e.g., as set forth in U.S. Pat. No. 7,531,637), a thymic stromal lymphopoietin (TSLP) antagonist (including, e.g., as set forth in US 2011/027468), interferon-gamma (IFNγ), antibiotics, topical corticosteroids, tacrolimus, pimecrolimus, cyclosporine, azathioprine, methotrexate, cromolyn sodium, proteinase inhibitors, systemic corticosteroids, systemic immunotherapy, anti-histamines, or combinations thereof.

In certain embodiments, the disclosed methods of administering a therapeutically effective amount of an IL-4/IL-13 pathway inhibitor and a therapeutically effective amount of a plasma cell ablating agent leads to reduction or abrogation of one or more symptoms or indications of an allergy as compared to an untreated subject or a subject treated with either inhibitor as monotherapy.

In certain embodiments, the disclosed methods lead to reduction, preferably total elimination of allergen-specific IgE in the treated subject. For instance, the disclosed methods of administering a therapeutically effective amount of an IL-4/IL-13 pathway inhibitor and a therapeutically effective amount of a plasma cell ablating agent promote at least about 50%, about 60%, about 70% or about 80% more decrease in the serum level of allergen-specific IgE in the treated subject as compared to an untreated subject or a subject treated with either inhibitor as monotherapy. In certain embodiments, the disclosed methods lead to complete elimination of allergen-specific serum IgE in the treated subject as compared to a subject treated with either agent as monotherapy.

According to certain embodiments, a subject may exhibit a decrease in the level of serum IgE specific to one or more allergens following administration of one or more doses of a IL-4/IL-13 pathway inhibitor (e.g., an anti-IL-4R antibody) in combination with one or more doses of a plasma cell ablating agent (e.g., an anti-BCMA/anti-CD3 bispecific antibody). For example, at about day 8, day 15, day 22, day 25, day 29, day 36, day 43, day 50, day 57, day 64, day 71, day 85, or day 112, following administration of one or more doses of an anti-IL-4R antibody (e.g., dupilumab) in combination with a plasma cell ablating agent, the subject, according to the present disclosure, may exhibit a decrease in allergen-specific IgE of about 50%, 55%, 60%, 65%, 70%, 75%, 80%, 85%, 90% or more from baseline (wherein “baseline” is defined as the level of allergen-specific IgE in the subject just prior to the first administration).

Methods for detecting and/or quantifying allergen-specific IgE or total IgE in the serum are known in the art; kits for measuring such are available from various commercial sources; and various commercial diagnostic laboratories offer services which provide measurement of such levels as well.

For example, Phadiatop™ is a commercially available variant of serum specific or antigen-specific IgE assay test that was introduced for the screening of allergic sensitization (Merrett et al 1987, Allergy 17: 409-416). The test provides for simultaneous testing for serum specific IgE to a mixture of relevant allergens causing common inhalant allergies. The test gives a qualitative result, either positive or negative depending upon a fluorescence response obtained. When a patient sample gives a fluorescence response higher than or equal to the reference, a positive test result is indicated. A patient sample with a lower fluorescence response indicates a negative test result. The present disclosure includes methods comprising selecting a subject who exhibits a positive test result and administering to the subject a therapeutically effective amount of an IL-4/IL-13 pathway inhibitor in combination with a plasma cell ablating agent.

In certain embodiments, the combination of administered therapeutic agents is safe and well tolerated by the subject such that there is no increase in adverse side effects as compared to a subject treated with either therapeutic agent as monotherapy.

### IL-4/IL-13 Pathway Inhibitors

The methods disclosed herein include administering a therapeutically effective amount of an IL-4/IL-13 pathway inhibitor to a subject in need thereof. As used herein, an “IL-4/IL-13 pathway inhibitor” (also referred to herein as an “IL-4/IL-13 pathway antagonist,” an “IL-4/IL-13 pathway blocker,” etc.) is any agent that inhibits or attenuates at least one of: (i) the binding of IL-4 and/or IL-13 to their respective receptors; (ii) signaling and/or activity of IL-4 and/or IL-13; and/or (iii) the downstream signaling/activity that results from binding of IL-4 and/or IL-13 to their respective receptors. Exemplary IL-4/IL-13 pathway inhibitors include, but are not limited to, anti-IL-4 antibodies (e.g., the antibodies disclosed in U.S. Pat. No. 7,740,843, and US Patent Application Publications 2010/0297110 and 2016/0207995), anti-IL-13 antibodies (e.g., the antibodies disclosed in U.S. Pat. Nos. 7,501,121, 7,674,459, 7,807,788, 7,910,708, 7,915,388, 7,935,343, 8,088,618, 8,691,233, and 9,605,065, US Patent Application Publications 2006/0073148 and 2008/0044420, and EP2627673B1), bispecific antibodies that bind to IL-4 and IL-13 (e.g., the antibodies disclosed in U.S. Pat. No. 8,388,965 and US Patent Application Publications 2011/0008345, 2013/0251718, and 2016/0207995), and IL-4 receptor (IL-4R) inhibitors (described below). The portions of the publications cited herein that identify IL-4/IL-13 pathway inhibitors are hereby incorporated by reference.

In some embodiments, the IL-4/IL-13 pathway inhibitor can be an antibody, a small molecule compound, a nucleic acid, a polypeptide, or a functional fragment or variant thereof. Non-limiting examples of suitable IL-4/IL-13 pathway inhibitor antibodies include anti-IL-4 antibodies, anti-IL-13 antibodies, and anti-IL-4/IL-13 bispecific antibodies, anti-IL-4R antibodies, and antigen-binding fragments of any of the foregoing. Other non-limiting examples of suitable IL-4/IL-13 pathway inhibitors include: RNAi molecules such as anti-IL-4 RNAi molecules and anti-IL-13 RNAi, antisense molecules such as anti-IL-4 antisense RNA and anti-IL-13 antisense RNA, and dominant negative proteins such as a dominant negative IL-4 protein, a dominant negative IL-13 protein.

As used herein, an “IL-4R inhibitor” (also referred to herein as an “IL-4/IL-13 pathway inhibitor,” an “IL-4Rα antagonist,” an “IL-4R blocker,” an “IL-4Rα blocker,” etc.) is any agent which binds to or interacts with IL-4Rα or an IL-4R ligand, and inhibits or attenuates the normal biological signaling function a type 1 and/or a type 2 IL-4 receptor. A type 1 IL-4 receptor is a dimeric receptor comprising an IL-4Rα chain and a γc chain. A type 2 IL-4 receptor is a dimeric receptor comprising an IL-4Rα chain and an IL-13Ra1 chain. Type 1 IL-4 receptors interact with and are stimulated by IL-4, while type 2 IL-4 receptors interact with and are stimulated by both IL-4 and IL-13. Thus, the IL-4R inhibitors that can be used in the methods of the present disclosure may function by blocking IL-4-mediated signaling, IL-13-mediated signaling, or both IL-4- and IL-13-mediated signaling. The IL-4R inhibitors of the present disclosure may thus prevent the interaction of IL-4 and/or IL-13 with a type 1 or type 2 receptor.

Non-limiting examples of categories of IL-4R inhibitors include IL-4 muteins (e.g., pitrakinra), small molecule IL-4R inhibitors, anti-IL-4R aptamers, peptide-based IL-4R inhibitors (e.g., “peptibody” molecules), “receptor-bodies” (e.g., engineered molecules comprising the ligand-binding domain of an IL-4R component), and antibodies or antigen-binding fragments of antibodies that specifically bind human IL-4Ra. As used herein, IL-4R inhibitors also include antigen-binding proteins that specifically bind IL-4 and/or IL-13.

Other non-limiting examples of suitable IL-4/IL-13 pathway inhibitors that can be used in the context of the present disclosure include, e.g., pitrakinra (AER-001; BAY-16-9996), aeroderm (AER-003), and the antibodies referred to and known in the art as dupilumab, pascolizumab, AMG-317, MILR1444A, CAT-354, QAX576, anrukinzumab (IMA-638), ISIS-369645 (AIR-645), IMA-026, APG-201, CNTO-607, MK-6105, MEDI9314, MEDI2045, tralokinumab, lebrikizumab, romilkimab, and DOM-0910.

### Anti-IL-4Rα Antibodies and Antigen-Binding Fragments Thereof

According to certain exemplary embodiments of the present disclosure, the IL-4/IL-13 pathway inhibitor is an anti-IL-4Rα antibody or antigen-binding fragment thereof. The term “antibody,” as used throughout the present disclosure, includes immunoglobulin molecules comprising four polypeptide chains, two heavy (H) chains and two light (L) chains inter-connected by disulfide bonds, as well as multimers thereof (e.g., IgM). In a typical antibody, each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments of the disclosure, the FRs of the antibody (or antigen-binding portion thereof) may be identical to the human germline sequences or may be naturally or artificially modified. An amino acid consensus sequence may be defined based on a side-by-side analysis of two or more CDRs.

The term “antibody,” as used throughout the present disclosure, includes antigen-binding fragments thereof—i.e., antigen-binding fragments of full antibody molecules. The terms “antigen-binding portion” of an antibody,” “antigen-binding fragment” of an antibody, and the like, as used throughout the present disclosure, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used throughout the present disclosure.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}—V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present disclosure include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2, (x) V_{L}-C_{H}3; (xi) V_{L}-C_{H}1-C_{H}2; (Xii) V_{L}-C_{H}1-C_{H}2-C_{H3}; (xiii) V_{L}-C_{H2}-C_{H3}; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present disclosure may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

The term “antibody,” as used throughout the present disclosure, also includes multispecific (e.g., bispecific) antibodies. A multispecific antibody or antigen-binding fragment of an antibody will typically comprise at least two different variable domains, wherein each variable domain is capable of specifically binding to a separate antigen or to a different epitope on the same antigen. Any multispecific antibody format may be adapted for use in the context of an antibody or antigen-binding fragment of an antibody of the present disclosure using routine techniques available in the art. For example, the present disclosure includes methods comprising the use of bispecific antibodies wherein one arm of an immunoglobulin is specific for IL-4Rα or a fragment thereof, and the other arm of the immunoglobulin is specific for a second therapeutic target or is conjugated to a therapeutic moiety. Exemplary bispecific formats that can be used in the context of the present disclosure include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED) body, leucine zipper, Duobody, IgG1/IgG2, dual acting Fab (DAF)-IgG, and Mab² bispecific formats (see, e.g., Klein et al. 2012, mAbs 4(6):653-663, and references cited therein, for a review of the foregoing formats). Bispecific antibodies can also be constructed using peptide/nucleic acid conjugation, e.g., wherein unnatural amino acids with orthogonal chemical reactivity are used to generate site-specific antibody-oligonucleotide conjugates which then self-assemble into multimeric complexes with defined composition, valency and geometry. (See, e.g., Kazane et al., J. Am. Chem. Soc., 2013, 135(1):340-46).

The antibodies used in the methods of the present disclosure may be human antibodies. The term “human antibody,” as used throughout the present disclosure, is intended to include antibodies having variable and constant regions derived from human germline immunoglobulin sequences. The human antibodies of the disclosure may nonetheless include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used throughout the present disclosure, is not intended to include antibodies in which CDR sequences derived from the germline of another mammalian species, such as a mouse, have been grafted onto human framework sequences.

The antibodies used in the methods of the present disclosure may be recombinant human antibodies. The term “recombinant human antibody,” as used throughout the present disclosure, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see, e.g., Taylor et al. (1992) Nucl. Acids Res., 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

According to certain embodiments, the antibodies used in the methods of the present disclosure specifically bind IL-4Ra. The term “specifically binds,” or the like, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. For example, in some embodiments, an antibody that “specifically binds” IL-4Ra, as used in the context of the present disclosure, includes antibodies that bind IL-4Rα or portion thereof with a K_{D} of less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM or less than about 0.5 nM, as measured in a surface plasmon resonance assay. An isolated antibody that specifically binds human IL-4Rα may, however, have cross-reactivity to other antigens, such as IL-4Rα molecules from other (non-human) species.

According to certain exemplary embodiments of the present disclosure, the IL-4/IL-13 pathway inhibitor is an anti-IL-4Rα antibody, or antigen-binding fragment thereof comprising a heavy chain variable region (HCVR), light chain variable region (LCVR), and/or complementarity determining regions (CDRs) comprising any of the amino acid sequences of the anti-IL-4R antibodies as set forth in U.S. Pat. No. 7,608,693, incorporated by reference herein. In certain exemplary embodiments, the anti-IL-4Rα antibody or antigen-binding fragment thereof that can be used in the context of the methods of the present disclosure comprises the heavy chain complementarity determining regions (HCDRs) of a heavy chain variable region (HCVR) comprising the amino acid sequence of SEQ ID NO: 1 and the light chain complementarity determining regions (LCDRs) of a light chain variable region (LCVR) comprising the amino acid sequence of SEQ ID NO: 2. According to certain embodiments, the anti-IL-4Rα antibody or antigen-binding fragment thereof comprises three HCDRs (HCDR1, HCDR2 and HCDR3) and three LCDRs (LCDR1, LCDR2 and LCDR3), wherein the HCDR1 comprises the amino acid sequence of SEQ ID NO: 3; the HCDR2 comprises the amino acid sequence of SEQ ID NO: 4; the HCDR3 comprises the amino acid sequence of SEQ ID NO: 5; the LCDR1 comprises the amino acid sequence of SEQ ID NO: 6; the LCDR2 comprises the amino acid sequence of SEQ ID NO: 7; and the LCDR3 comprises the amino acid sequence of SEQ ID NO: 8. In yet other embodiments, the anti-IL-4R antibody or antigen-binding fragment thereof comprises an HCVR comprising SEQ ID NO: 1 and an LCVR comprising SEQ ID NO: 2. In certain embodiments, the methods of the present disclosure comprise the use of an anti-IL-4R antibody, wherein the antibody comprises a heavy chain comprising the amino acid sequence of SEQ ID NO: 9. In some embodiments, the anti-IL-4R antibody comprises a light chain comprising the amino acid sequence of SEQ ID NO: 10. An exemplary antibody comprising a heavy chain comprising the amino acid sequence of SEQ ID NO: 9 and a light chain comprising the amino acid sequence of SEQ ID NO: 10 is the fully human anti-IL-4R antibody known as dupilumab (DUPIXENT™). According to certain exemplary embodiments, the methods of the present disclosure comprise the use of dupilumab, or a bioequivalent thereof. The term “bioequivalent” with respect to dupilumab refers to anti-IL-4R antibodies or IL-4R-binding proteins or fragments thereof that are pharmaceutical equivalents or pharmaceutical alternatives having a rate and/or extent of absorption that does not show a significant difference with that of dupilumab when administered at the same molar dose under similar experimental conditions, either single dose or multiple dose. In the context of the present disclosure, the term refers to antigen-binding proteins that bind to IL-4R which do not have clinically meaningful differences with dupilumab in their safety, purity and/or potency.

According to certain embodiments of the present disclosure, the anti-human IL-4R antibody or antigen-binding fragment thereof comprises a HCVR having at least 90%, 91%, 92%, 93%, 94%, 95%, 96%, 97%, 98%, or 99% sequence identity to SEQ ID NO: 1. In some embodiments, the anti-human IL-4R antibody or antigen-binding fragment thereof comprises three HCDRs (HCDR1, HCDR2 and HCDR3) comprising the amino acid sequences of SEQ ID NOs:3, 4, and 5, respectively, and comprises a HCVR having at least 90%, at least 91%, at least 92%, at least 93%, at least 94%, at least 95%, at least 96%, at least 97%, at least 98%, or at least 99% sequence identity to SEQ ID NO:1.

According to certain embodiments of the present disclosure, the anti-human IL-4R antibody or antigen-binding fragment thereof comprises a LCVR having at least 90%, 91%, 92%, 93%, 94%, 95%, 96%, 97%, 98%, or 99% sequence identity to SEQ ID NO: 2. In some embodiments, the anti-human IL-4R antibody or antigen-binding fragment thereof comprises three LCDRs (LCDR1, LCDR2 and LCDR3) comprising the amino acid sequences of SEQ ID NOs:6, 7, and 8, respectively, and comprises a LCVR having at least 90%, at least 91%, at least 92%, at least 93%, at least 94%, at least 95%, at least 96%, at least 97%, at least 98%, or at least 99% sequence identity to SEQ ID NO:2.

According to certain embodiments of the present disclosure, the anti-human IL-4R antibody or antigen-binding fragment thereof comprises a HCVR comprising an amino acid sequence of SEQ ID NO: 1 having no more than 5 amino acid substitutions. According to certain embodiments of the present disclosure, the anti-human IL-4R antibody or antigen-binding fragment thereof comprises a LCVR comprising an amino acid sequence of SEQ ID NO: 2 having no more than 2 amino acid substitutions.

In some embodiments, the anti-human IL-4R antibody or antigen-binding fragment thereof comprises a HCVR having 90%, 91%, 92%, 93%, 94%, 95%, 96%, 97%, 98%, or 99% sequence identity to SEQ ID NO: 1 and a LCVR having 90%, 91%, 92%, 93%, 94%, 95%, 96%, 97%, 98%, or 99% sequence identity to SEQ ID NO: 2. In some embodiments, the anti-human IL-4R antibody or antigen-binding fragment thereof comprises (a) an HCDR1, HCDR2 and HCDR3 comprising the amino acid sequences of SEQ ID NOs:3, 4, and 5, respectively, and a HCVR having at least 90%, at least 91%, at least 92%, at least 93%, at least 94%, at least 95%, at least 96%, at least 97%, at least 98%, or at least 99% sequence identity to SEQ ID NO:1; and (b) an LCDR1, LCDR2 and LCDR3 comprising the amino acid sequences of SEQ ID NOs:6, 7, and 8, respectively, and a LCVR having at least 90%, at least 91%, at least 92%, at least 93%, at least 94%, at least 95%, at least 96%, at least 97%, at least 98%, or at least 99% sequence identity to SEQ ID NO:2.

Sequence identity may be measured by methods known in the art (e.g., GAP, BESTFIT, and BLAST).

The present disclosure also includes use of anti-IL-4R antibodies in methods to treat allergy or to eliminate allergen-specific IgE in a subject, wherein the anti-IL-4R antibodies comprise variants of any of the HCVR, LCVR and/or CDR amino acid sequences disclosed herein having one or more conservative amino acid substitutions. For example, the present disclosure includes use of anti-IL-4R antibodies having HCVR, LCVR and/or CDR amino acid sequences with, e.g., 10 or fewer, 8 or fewer, 6 or fewer, 4 or fewer, etc. conservative amino acid substitutions relative to any of the HCVR, LCVR and/or CDR amino acid sequences disclosed herein. In some embodiments, the disclosure includes use of an anti-IL-4R antibody having HCVR, LCVR and/or CDR amino acid sequences with 1, 2, 3, or 4 conservative amino acid substitutions relative to any of the HCVR, LCVR and/or CDR amino acid sequences disclosed herein.

Other anti-IL-4Rα antibodies that can be used in the context of the methods of the present disclosure include, e.g., the antibody referred to and known in the art as AMG317 (Corren et al., 2010, Am J Respir Crit Care Med., 181(8):788-796), or MEDI 9314, or any of the anti-IL-4Rα antibodies as set forth in U.S. Pat. Nos. 7,186,809, 7,605,237, 7,638,606, 8,092,804, 8,679,487, or U.S. Pat. No. 8,877,189. The portions of the publications cited herein that identify anti-IL-4Rα antibodies are hereby incorporated by reference.

The anti-IL-4Rα antibodies used in the context of the methods of the present disclosure may have pH-dependent binding characteristics. For example, an anti-IL-4Rα antibody for use in the methods of the present disclosure may exhibit reduced binding to IL-4Rα at acidic pH as compared to neutral pH. Alternatively, an anti-IL-4Rα antibody of the present disclosure may exhibit enhanced binding to its antigen at acidic pH as compared to neutral pH. The expression “acidic pH” includes pH values less than about 6.2, e.g., about 6.0, 5.95, 5.9, 5.85, 5.8, 5.75, 5.7, 5.65, 5.6, 5.55, 5.5, 5.45, 5.4, 5.35, 5.3, 5.25, 5.2, 5.15, 5.1, 5.05, 5.0, or less. As used throughout the present disclosure, the expression “neutral pH” means a pH of about 7.0 to about 7.4. The expression “neutral pH” includes pH values of about 7.0, 7.05, 7.1, 7.15, 7.2, 7.25, 7.3, 7.35, and 7.4.

In certain instances, “reduced binding to IL-4Rα at acidic pH as compared to neutral pH” is expressed in terms of a ratio of the K_{D} value of the antibody binding to IL-4Rα at acidic pH to the K_{D} value of the antibody binding to IL-4Rα at neutral pH (or vice versa). For example, an antibody or antigen-binding fragment thereof may be regarded as exhibiting “reduced binding to IL-4Rα at acidic pH as compared to neutral pH” for purposes of the present disclosure if the antibody or antigen-binding fragment thereof exhibits an acidic/neutral K_{D} ratio of about 3.0 or greater. In certain exemplary embodiments, the acidic/neutral K_{D} ratio for an antibody or antigen-binding fragment of the present disclosure can be about 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 20.0, 25.0, 30.0, 40.0, 50.0, 60.0, 70.0, 100.0, or greater.

Antibodies with pH-dependent binding characteristics may be obtained, e.g., by screening a population of antibodies for reduced (or enhanced) binding to a particular antigen at acidic pH as compared to neutral pH. Additionally, modifications of the antigen-binding domain at the amino acid level may yield antibodies with pH-dependent characteristics. For example, by substituting one or more amino acids of an antigen-binding domain (e.g., within a CDR) with a histidine residue, an antibody with reduced antigen-binding at acidic pH relative to neutral pH may be obtained. As used throughout the present disclosure, the expression “acidic pH” means a pH of 6.0 or less.

### Plasma Cell Ablating Agents

The methods disclosed herein include administering a therapeutically effective amount of a plasma cell ablating agent to a subject in need thereof. As used herein, a “plasma cell ablating agent” refers to any molecule capable of specifically binding to a surface antigen on plasma cells and killing or ablating said plasma cell. In some embodiments, the plasma cell ablating agent can be an antibody, a small molecule compound, a nucleic acid, a polypeptide, or a functional fragment or variant thereof. In the context of the present disclosure, a plasma cell ablating agent is used in combination with an IL-4/IL-13 pathway inhibitor in the disclosed methods.

Non-limiting examples of suitable plasma cell ablating agents include BCMA targeting agents (described elsewhere herein), proteasome inhibitors [e.g., bortezomib (Velcade), carfilzomib (Kyprolis), ixazornib (Ninlaro)], histone deacetylase inhibitors [e.g., panobinostat (Farydak)], B-cell activating factor (BAFF; also referred to as BLyS, TALL-1, or CD257) inhibitors (e.g., anti-BAFF antibodies such as belimumab, tabalumab, AMG570; or anti-BAFF receptor antibodies such as ianalumab), and A proliferation-inducing ligand (APRIL; also referred to as TNFSF13 or CD256) inhibitors (e.g., anti-APRIL antibodies such as BION-1301 or VIS624).

### BCMA Targeting Agents

According to certain exemplary embodiments, the plasma cell ablating agents used in the methods disclosed herein are BCMA targeting agents.

As used herein, the term “BCMA targeting agent” refers to any molecule capable of binding specifically to BCMA that is expressed on the surface of a cell in a subject, thus targeting said cell for destruction. BCMA is expressed exclusively in B-cell lineage cells, particularly in the interfollicular region of the germinal center as well as on plasmablasts and differentiated plasma cells. BCMA is selectively induced during plasma cell differentiation and is required for optimal survival of long-lived plasma cells in the bone marrow. Thus, a BCMA targeting agent binds to BCMA expressed on plasma cell surface and mediates killing or ablation of cells that express BCMA (plasma cell ablation). In the context of the present disclosure, in some embodiments a BCMA targeting agent comprises a binding moiety that binds to plasma cell-surface-expressed BCMA (an antigen-binding moiety or antigen-binding fragment thereof) and a moiety that facilitates killing of said plasma cell. In certain embodiments, the plasma cell-surface-expressed BCMA-binding moiety is an antibody or antigen-binding fragment thereof that binds specifically to BCMA. Such a BCMA-binding moiety is linked (e.g., covalently bound) to a moiety that facilitates killing or destruction of the targeted plasma cell. The moiety that facilitates targeted killing of the bound plasma cell may be a molecule that directly kills the targeted cell (e.g., a cytotoxic agent) or may be a protein or fragment thereof that mediates killing of the targeted cell by an immune cell, e.g., a T-cell. In the context of the present disclosure, the term “BCMA targeting agent” includes, but is not limited to, anti-BCMA antibodies that are conjugated to a therapeutic agent such as a cytotoxic drug (“BCMA ADC” or “anti-BCMA ADC”), chimeric antigenic receptors (CARs) that bind specifically to BCMA, (“BCMA CAR” or “anti-BCMA CAR”) and anti-BCMA/anti-CD3 bispecific antibodies.

According to certain embodiments, the BCMA targeting agent used in the context of the disclosed methods is an antibody-drug conjugate (ADC) comprising an anti-BCMA antibody and a cytotoxic drug. In some embodiments, the anti-BCMA antibody or antigen-binding fragment thereof and the cytotoxic agent are covalently attached via a linker. In general terms, the ADCs comprise: A-[L-P]_{y}, in which A is an antigen-binding molecule, e.g., an anti-BCMA antibody, or a fragment thereof, L is a linker, P is the payload or therapeutic moiety (e.g., cytotoxic agent), and y is an integer from 1 to 30. Examples of suitable cytotoxic agents and chemotherapeutic agents for forming ADCs are known in the art. Non-limiting examples of suitable cytotoxic agents that can be conjugated to anti-BCMA antibodies for use in the disclosed methods are auristatin such as monomethyl auristatin E (MMAE) or monomethyl auristatin F (MMAF), a tubulysin such as TUB-OH or TUB-OMOM, a tomaymycin derivative, a dolastatin derivative, or a maytansinoid such as DM1 or DM4. According to certain exemplary embodiments, the present disclosure includes use of anti-BCMA ADCs in methods to treat allergies, wherein the anti-BCMA ADCs comprise any of the HCVR, LCVR and/or CDR amino acid sequences disclosed elsewhere herein.

The present disclosure also includes use of anti-BCMA ADCs in methods to treat allergy, wherein the antibodies comprise variants of any of the HCVR, LCVR and/or CDR amino acid sequences disclosed herein having one or more conservative amino acid substitutions. For example, the present disclosure includes use of antibodies having HCVR, LCVR and/or CDR amino acid sequences with, e.g., 10 or fewer, 8 or fewer, 6 or fewer, 4 or fewer, etc. conservative amino acid substitutions relative to any of the HCVR, LCVR and/or CDR amino acid sequences disclosed herein. In some embodiments, the disclosure includes use of an antibody having HCVR, LCVR and/or CDR amino acid sequences with 1, 2, 3, or 4 conservative amino acid substitutions relative to any of the HCVR, LCVR and/or CDR amino acid sequences disclosed herein.

Other anti-BCMA ADCs that can be used in the context of the methods of the present disclosure include, e.g., the ADCs referred to and known in the art as belantamab mafodotin, GSK2857916, AMG224, HDP-101, MED12228, and TBL-CLN1, or any of the anti-BCMA ADCs set forth, e.g., in patent publications WO2011/108008, WO2014/089335, WO2017/093942, WO2017/143069, WO2019/025983. The portions of the publications cited herein that identify anti-BCMA ADCs are hereby incorporated by reference.

According to certain embodiments, the BCMA targeting agent used in the context of the disclosed methods is a chimeric antigen receptor (CAR) that binds specifically to BCMA (“BCMA CAR”). The term “chimeric antigen receptor” (CAR) refers to molecules that combine a binding domain against a component present on the target cell, for example an antibody-based specificity for a desired antigen (e.g., BCMA on plasma cell) with a T cell receptor-activating intracellular domain to generate a chimeric protein that exhibits a specific anti-target cellular immune activity. Generally, CARs consist of an extracellular single chain antibody-binding domain (scFv) fused to the intracellular signaling domain of the T cell antigen receptor complex zeta chain, and have the ability, when expressed in T cells, to redirect antigen recognition based on the monoclonal antibody's specificity. In certain embodiments, the BCMA CAR or antigen-binding fragment thereof comprises a heavy chain variable region (HCVR), light chain variable region (LCVR), and/or complementarity determining regions (CDRs) comprising the amino acid sequences of any of the antibodies set forth in US provisional patent application U.S. Ser. No. 62/700,615, filed on Jul. 19, 2018, or in International Patent Application No. PCT/US2019/042452, which are hereby incorporated by reference in its entirety. According to certain exemplary embodiments, the present disclosure includes use of anti-BCMA CARs in methods to treat allergies, wherein the anti-BCMA CARs comprise any of the HCVR, LCVR and/or CDR amino acid sequences disclosed elsewhere herein.

The present disclosure also includes use of anti-BCMA CARs in methods to treat allergy, wherein the CARs comprise variants of any of the HCVR, LCVR and/or CDR amino acid sequences disclosed herein having one or more conservative amino acid substitutions. For example, the present disclosure includes use of anti-BCMA CARs having HCVR, LCVR and/or CDR amino acid sequences with, e.g., 10 or fewer, 8 or fewer, 6 or fewer, 4 or fewer, etc. conservative amino acid substitutions relative to any of the HCVR, LCVR and/or CDR amino acid sequences disclosed herein. In some embodiments, the disclosure includes use of an anti-BCMA CAR having HCVR, LCVR and/or CDR amino acid sequences with 1, 2, 3, or 4 conservative amino acid substitutions relative to any of the HCVR, LCVR and/or CDR amino acid sequences disclosed herein.

Other anti-BCMA CARs that can be used in the context of the methods of the present disclosure include, e.g., the CARs referred to and known in the art as bb2121, LCAR-B38M, and 4C8A, or any of the anti-BCMA CARs set forth, e.g., in patent publications WO2015/052538, WO2015/052536, WO2016/094304, WO2016/166630, WO2016/151315, WO2016/130598, WO2017/183418, WO2017/173256, WO2017211900, WO2017/130223, WO2018/229492, WO2018/085690, WO2018/151836, WO2018/028647, WO2019/006072. The portions of the publications cited herein that identify anti-BCMA CARs are hereby incorporated by reference.

According to certain exemplary embodiments, the BCMA targeting agent used in the context of the disclosed methods is an anti-BCMA/anti-CD3 bispecific antibody (also referred to herein as an “anti-BCMA×anti-CD3 bispecific antibody”). The anti-BCMA/anti-CD3 bispecific antibodies are useful for specific targeting and T-cell-mediated killing of cells that express BCMA. The terms “antibody,” “antigen-binding fragment,” “human antibody,” “recombinant antibody,” and other related terminology are defined above. In the context of anti-BCMA/anti-CD3 antibodies and antigen-binding fragments thereof, the present disclosure includes the use of bispecific antibodies wherein one arm of an immunoglobulin is specific for BCMA or a fragment thereof, and the other arm of the immunoglobulin is specific for a second therapeutic target (e.g., CD3 on T-cells). Exemplary bispecific formats that can be used in the context of the present disclosure include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED) body, leucine zipper, Duobody, IgG1/IgG2, dual acting Fab (DAF)-IgG, and Mab² bispecific formats (see, e.g., Klein et al. 2012, mAbs 4(6):653-663, and references cited therein, for a review of the foregoing formats). Bispecific antibodies can also be constructed using peptide/nucleic acid conjugation, e.g., wherein unnatural amino acids with orthogonal chemical reactivity are used to generate site-specific antibody-oligonucleotide conjugates which then self-assemble into multimeric complexes with defined composition, valency and geometry. (See, e.g., Kazane et al., J. Am. Chem. Soc., 2013, 135(1):340-46).

The term “specifically binds,” or the like, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. For example, an antibody that “specifically binds” BCMA, as used in the context of the present disclosure, includes antibodies that bind BCMA or a portion thereof with a K_{D} of less than about 100 nM, less than about 50 nM, less than about 20 nM, less than about 10 nM, less than about 5 nM, less than about 1 nM, less than about 500 pM, less than about 200 pM, less than about 100 pM, or less than about 50 pM, as measured in a surface plasmon resonance assay. An isolated antibody that specifically binds human BCMA may, however, have cross-reactivity to other antigens, such as BCMA molecules from other (non-human) species.

According to certain exemplary embodiments, the anti-BCMA/anti-CD3 bispecific antibody, or antigen-binding fragment thereof comprises a heavy chain variable region (HCVR), light chain variable region (LCVR), and/or complementarity determining regions (CDRs) comprising the amino acid sequences of any of the antibodies set forth in US provisional patent application U.S. Ser. No. 62/793,645, filed on Jan. 17, 2019, or in International Patent Application No. PCT/US2019/042447, which are hereby incorporated by reference in its entirety. In certain exemplary embodiments, the anti-BCMA/anti-CD3 bispecific antibody or antigen-binding fragment thereof that can be used in the context of the present disclosure comprises: (a) a first antigen binding domain that binds specifically to BCMA; and (b) a second antigen-binding domain that binds specifically to CD3. In one embodiment, the first antigen-binding domain comprises the heavy chain complementarity determining regions (HCDRs) of a heavy chain variable region (HCVR) comprising the amino acid sequence of SEQ ID NO: 12 and the light chain complementarity determining regions (LCDRs) of a light chain variable region (LCVR) comprising the amino acid sequence of SEQ ID NO: 20. According to certain embodiments, the first antigen-binding domain comprises three HCDRs (HCDR1, HCDR2 and HCDR3) and three LCDRs (LCDR1, LCDR2 and LCDR3), wherein the HCDR1 comprises the amino acid sequence of SEQ ID NO: 14; the HCDR2 comprises the amino acid sequence of SEQ ID NO: 16; the HCDR3 comprises the amino acid sequence of SEQ ID NO: 18; the LCDR1 comprises the amino acid sequence of SEQ ID NO: 22; the LCDR2 comprises the amino acid sequence of SEQ ID NO: 24; and the LCDR3 comprises the amino acid sequence of SEQ ID NO: 26. In one embodiment, the second antigen-binding domain comprises the heavy chain complementarity determining regions (HCDRs) of a heavy chain variable region (HCVR) comprising the amino acid sequence of SEQ ID NO: 28 or SEQ ID NO: 36 and the light chain complementarity determining regions (LCDRs) of a light chain variable region (LCVR) comprising the amino acid sequence of SEQ ID NO: 20. In one embodiment, the second antigen-binding domain comprises three HCDRs (HCDR1, HCDR2 and HCDR3) and three LCDRs (LCDR1, LCDR2 and LCDR3), wherein the HCDR1 comprises the amino acid sequence of SEQ ID NO: 30 or 38; the HCDR2 comprises the amino acid sequence of SEQ ID NO: 32 or 40; the HCDR3 comprises the amino acid sequence of SEQ ID NO: 34 or 42; the LCDR1 comprises the amino acid sequence of SEQ ID NO: 22; the LCDR2 comprises the amino acid sequence of SEQ ID NO: 24; and the LCDR3 comprises the amino acid sequence of SEQ ID NO: 26.

In one embodiment, the anti-BCMA/anti-CD3 bispecific antibody or antigen-binding fragment thereof comprises: (a) a first antigen-binding domain that comprises HCDR1, HCDR2, and HCDR3 domains, respectively, comprising the amino acid sequences of SEQ ID NOs: 14, 16, and 18, and LCDR1, LCDR2, and LCDR3 domains, respectively, comprising the amino acid sequences of SEQ ID NOs: 22, 24, and 26; and (b) a second antigen binding domain that comprises HCDR1, HCDR2, and HCDR3 domains, respectively, comprising the amino acid sequences of SEQ ID NOs: 30, 32, and 34, and LCDR1, LCDR2, and LCDR3 domains, respectively, comprising the amino acid sequences of SEQ ID NOs: 22, 24, and 26. In one embodiments, the anti-BCMA/anti-CD3 bispecific antibody or antigen-binding fragment thereof comprises: (a) a first antigen-binding domain that comprises a HCVR comprising the amino acid sequence of SEQ ID NO:12 and a LCVR comprising the amino acid sequence of SEQ ID NO:20; and (b) a second antigen-binding domain that comprises a HCVR comprising the amino acid sequence of SEQ ID NO:28 and a LCVR comprising the amino acid sequence of SEQ ID NO:20.

In one embodiment, the anti-BCMA/anti-CD3 bispecific antibody or antigen-binding fragment thereof comprises: (a) a first antigen-binding domain that comprises HCDR1, HCDR2, and HCDR3 domains, respectively, comprising the amino acid sequences of SEQ ID NOs: 14, 16, and 18, and LCDR1, LCDR2, and LCDR3 domains, respectively, comprising the amino acid sequences of SEQ ID NOs: 22, 24, and 26; and (b) a second antigen binding domain that comprises HCDR1, HCDR2, and HCDR3 domains, respectively, comprising the amino acid sequences of SEQ ID NOs: 38, 40, and 42, and LCDR1, L CDR2, and LCDR3 domains, respectively, comprising the amino acid sequences of SEQ ID NOs: 22, 24, and 26. In one embodiments, the anti-BCMA/anti-CD3 bispecific antibody or antigen-binding fragment thereof comprises: (a) a first antigen-binding domain that comprises a HCVR comprising the amino acid sequence of SEQ ID NO:12 and a LCVR comprising the amino acid sequence of SEQ ID NO:20; and (b) a second antigen-binding domain that comprises a HCVR comprising the amino acid sequence of SEQ ID NO:36 and a LCVR comprising the amino acid sequence of SEQ ID NO:20.

Exemplary anti-BCMA/anti-CD3 bispecific antibodies include the fully human bispecific antibodies known as REGN5458 and REGN5459. According to certain exemplary embodiments, the methods of the present disclosure comprise the use of REGN5458 or REGN5459, or a bioequivalent thereof. As used herein, the term “bioequivalent” with respect to anti-BCMA/anti-CD3 antibodies refers to antibodies or BCMA/CD3-binding proteins or fragments thereof that are pharmaceutical equivalents or pharmaceutical alternatives having a rate and/or extent of absorption that does not show a significant difference with that of a reference antibody (e.g., REGN5458 or REGN5459) when administered at the same molar dose under similar experimental conditions, either single dose or multiple dose; the term “bioequivalent” also includes antigen-binding proteins that bind to BCMA/CD3 and do not have clinically meaningful differences with the reference antibody (e.g., REGN5458 or REGN5459) with respect to safety, purity and/or potency.

In some embodiments, the anti-BCMA/anti-CD3 bispecific antibody or antigen-binding fragment thereof comprises: (a) a first antigen-binding domain that comprises a HCVR having at least 90%, 91%, 92%, 93%, 94%, 95%, 96%, 97%, 98%, or 99% sequence identity to the amino acid sequence of SEQ ID NO: 12 and a LCVR having at least 90%, 91%, 92%, 93%, 94%, 95%, 96%, 97%, 98%, or 99% sequence identity to the amino acid sequence of SEQ ID NO:20; and (b) a second antigen-binding domain that comprises a HCVR having at least 90%, 91%, 92%, 93%, 94%, 95%, 96%, 97%, 98%, or 99% sequence identity to the amino acid sequence of SEQ ID NO: 28 and a LCVR having at least 90%, 91%, 92%, 93%, 94%, 95%, 96%, 97%, 98%, or 99% sequence identity to the amino acid sequence of SEQ ID NO:20. In some embodiments, the anti-BCMA/anti-CD3 bispecific antibody or antigen-binding fragment thereof comprises: (a) a first antigen-binding domain that comprises three HCDRs (HCDR1, HCDR2 and HCDR3) comprising the amino acid sequences of SEQ ID NOs:14, 16, and 18, respectively, and a HCVR having at least 90%, 91%, 92%, 93%, 94%, 95%, 96%, 97%, 98%, or 99% sequence identity to the amino acid sequence of SEQ ID NO: 12, and comprises three LCDRs (LCDR1, LCDR2 and LCDR3) comprising the amino acid sequences of SEQ ID NOs:22, 24, and 26, respectively, and a LCVR having at least 90%, 91%, 92%, 93%, 94%, 95%, 96%, 97%, 98%, or 99% sequence identity to the amino acid sequence of SEQ ID NO:20; and (b) a second antigen-binding domain that comprises three HCDRs (HCDR1, HCDR2 and HCDR3) comprising the amino acid sequences of SEQ ID NOs:30, 32, and 34, respectively, and a HCVR having at least 90%, 91%, 92%, 93%, 94%, 95%, 96%, 97%, 98%, or 99% sequence identity to the amino acid sequence of SEQ ID NO: 28, and comprises three LCDRs (LCDR1, LCDR2 and LCDR3) comprising the amino acid sequences of SEQ ID NOs:22, 24, and 26, respectively, and a LCVR having at least 90%, 91%, 92%, 93%, 94%, 95%, 96%, 97%, 98%, or 99% sequence identity to the amino acid sequence of SEQ ID NO:20.

In some embodiments, the anti-BCMA/anti-CD3 bispecific antibody or antigen-binding fragment thereof comprises: (a) a first antigen-binding domain that comprises a HCVR having at least 90%, 91%, 92%, 93%, 94%, 95%, 96%, 97%, 98%, or 99% sequence identity to the amino acid sequence of SEQ ID NO: 12 and a LCVR having at least 90%, 91%, 92%, 93%, 94%, 95%, 96%, 97%, 98%, or 99% sequence identity to the amino acid sequence of SEQ ID NO:20; and (b) a second antigen-binding domain that comprises a HCVR having at least 90%, 91%, 92%, 93%, 94%, 95%, 96%, 97%, 98%, or 99% sequence identity to the amino acid sequence of SEQ ID NO: 36 and a LCVR having at least 90%, 91%, 92%, 93%, 94%, 95%, 96%, 97%, 98%, or 99% sequence identity to the amino acid sequence of SEQ ID NO:20. In some embodiments, the anti-BCMA/anti-CD3 bispecific antibody or antigen-binding fragment thereof comprises: (a) a first antigen-binding domain that comprises three HCDRs (HCDR1, HCDR2 and HCDR3) comprising the amino acid sequences of SEQ ID NOs:14, 16, and 18, respectively, and a HCVR having at least 90%, 91%, 92%, 93%, 94%, 95%, 96%, 97%, 98%, or 99% sequence identity to the amino acid sequence of SEQ ID NO: 12, and comprises three LCDRs (LCDR1, LCDR2 and LCDR3) comprising the amino acid sequences of SEQ ID NOs:22, 24, and 26, respectively, and a LCVR having at least 90%, 91%, 92%, 93%, 94%, 95%, 96%, 97%, 98%, or 99% sequence identity to the amino acid sequence of SEQ ID NO:20; and (b) a second antigen-binding domain that comprises three HCDRs (HCDR1, HCDR2 and HCDR3) comprising the amino acid sequences of SEQ ID NOs:38, 40, and 42, respectively, and a HCVR having at least 90%, 91%, 92%, 93%, 94%, 95%, 96%, 97%, 98%, or 99% sequence identity to the amino acid sequence of SEQ ID NO: 36, and comprises three LCDRs (LCDR1, LCDR2 and LCDR3) comprising the amino acid sequences of SEQ ID NOs:22, 24, and 26, respectively, and a LCVR having at least 90%, 91%, 92%, 93%, 94%, 95%, 96%, 97%, 98%, or 99% sequence identity to the amino acid sequence of SEQ ID NO:20.

The present disclosure also includes use of anti-BCMA/anti-CD3 antibodies in methods to treat allergy, wherein the antibodies comprise variants of any of the HCVR, LCVR and/or CDR amino acid sequences disclosed herein having one or more conservative amino acid substitutions. For example, the present disclosure includes use of anti-BCMA/anti-CD3 antibodies having HCVR, LCVR and/or CDR amino acid sequences with, e.g., 10 or fewer, 8 or fewer, 6 or fewer, 4 or fewer, etc. conservative amino acid substitutions relative to any of the HCVR, LCVR and/or CDR amino acid sequences disclosed herein. In some embodiments, the disclosure includes use of an anti-BCMA/anti-CD3 antibody having HCVR, LCVR and/or CDR amino acid sequences with 1, 2, 3, or 4 conservative amino acid substitutions relative to any of the HCVR, LCVR and/or CDR amino acid sequences disclosed herein.

Other anti-BCMA/anti-CD3 antibodies that can be used in the context of the methods of the present disclosure include, e.g., the antibodies referred to and known in the art as AMG420, AMG701, CC-93269, EM801, JNJ-64007957, and TNB384B, or any of the anti-BCMA/anti-CD3 antibodies set forth, e.g., in patent publications WO2013/072415, WO2014/140248, WO2014/122144, WO2016/166629, WO2016/079177, WO2016/020332, WO2017031104, WO2017/223111, WO2017/134134, WO2018/083204, WO2018/201051. The portions of the publications cited herein that identify anti-BCMA/anti-CD3 antibodies are hereby incorporated by reference.

IgE Depletion in Combination with Allergen Immunotherapy

The present disclosure also provides methods for enhancing the efficacy and/or tolerability of an immunotherapy regimen (e.g., an allergen-specific immunotherapy regimen) in a subject having an allergy. In some embodiments, the method comprises administering to a subject having an allergy an IL-4/IL-13 pathway inhibitor (such as an anti-IL-4R antibody) and a plasma cell ablating agent (such as an anti-BCMA/anti-CD3 antibody) prior to or concurrent with the immunotherapy regimen.

In some embodiments, the subject to be treated has a food allergy. For example, in some embodiments, the subject has an allergy to milk, a dairy product, egg, celery, sesame, wheat, meat, fruit, soy, fish, shellfish, a sugar, peanut, a legume, a tree nut, or a combination thereof. In some embodiments, the subject has a peanut allergy. In some embodiments, the subject to be treated has a non-food allergy (e.g., an allergy to an environmental allergen). For example, in some embodiments, the subject has an allergy to a non-food allergen selected from the group consisting of dust, dust mite, pollen, insect venom, mold, animal fur, animal dander, wool, latex, a metal, a household cleaner, a detergent, medication, cosmetics, perfumes, a drug such as penicillin, sulfonamides, or salicylate, therapeutic monoclonal antibodies (e.g., cetuximab), ragweed, grass and birch. In some embodiments, the subject to be treated has a severe allergy (e.g., a severe food allergy or a severe non-food allergy).

As used herein, “allergen-specific immunotherapy” refers to the repeated administration of an allergen (e.g., an allergen as disclosed herein) to a subject over time as a means for treating or preventing allergies and allergic reactions, or to reduce to eliminate allergic responses. In some embodiments, the allergen-specific immunotherapy regimen comprises oral immunotherapy. In some embodiments, the allergen-specific immunotherapy regimen comprises subcutaneous immunotherapy. In some embodiments, the allergen-specific immunotherapy regimen comprises sublingual immunotherapy. In general, the immunotherapy regimen can be a “conventional” immunotherapy regimen or an “accelerated” immunotherapy regimen. Typically, in a conventional immunotherapy regimen, increasing doses of the allergen (also referred to as “up-titration”) are administered to the patient at weekly intervals over the course of several weeks to months (e.g., over 1, 2, 3, 4, 5, 6, 7, 8, 9, 10, 11, 12 months or longer), under tightly monitored medical supervision, followed by a maintenance regimen, which typically comprises administering one or more doses of the allergen at the highest dose administered during the up-titration regimen. In an accelerated immunotherapy regimen, the up-titration schedule is accelerated as compared to conventional immunotherapy. Examples of accelerated immunotherapy include “rush” immunotherapy and “cluster” immunotherapy. In rush immunotherapy, typically increasing dosages of the allergen are administered per day over several consecutive days (e.g., over 2 days, 3 days, 4 days, 5 days, 6 days, or one week) until the maximum tolerated dose is reached. In cluster immunotherapy, typically several (e.g., 2-3) increasing dosages of the allergen are administered in a single day, over nonconsecutive days until the maximum tolerated dose is reached, usually within 4 to 8 weeks.

In some embodiments, an IL-4/IL-13 pathway inhibitor and a plasma cell ablating agent are administered prior to or concurrent with an allergen-specific immunotherapy regimen as disclosed herein (e.g., oral, sublingual, or subcutaneous immunotherapy, which can be conventional or accelerated immunotherapy). In some embodiments, the plasma cell ablating agent (e.g., 1, 2, 3, 4, 5 or more doses) is administered prior to the onset of the immunotherapy regimen. In some embodiments, the IL-4/IL-13 pathway inhibitor (e.g., 1, 2, 3, 4, 5 or more doses) is administered prior to the onset of the immunotherapy regimen. In some embodiments, at least one dose of each of the plasma cell ablating agent and the IL-4/IL-13 pathway inhibitor are administered prior to the onset of the immunotherapy regimen. In some embodiments, the IL-4/IL-13 pathway inhibitor is administered concurrent with the immunotherapy regimen. In some embodiments, the plasma cell ablating agent is administered concurrent with the immunotherapy regimen.

### Pharmaceutical Compositions and Administration

The disclosed methods comprise administering an IL-4/IL-13 pathway inhibitor in combination with a plasma cell ablating agent to a subject in need thereof, wherein the inhibitors are contained within separate pharmaceutical compositions or a combined (single) pharmaceutical composition. The pharmaceutical compositions of the disclosure may be formulated with pharmaceutically acceptable carriers, excipients, and other agents that provide suitable transfer, delivery, tolerance, and the like. A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al., 1998, J Pharm Sci Technol, 52:238-311.

In certain embodiments, the pharmaceutical compositions of the disclosure comprise a therapeutically effective amount of an IL-4/IL-13 pathway inhibitor (such as an anti-IL-4R antibody) and/or a therapeutically effective amount of a plasma cell ablating agent (such as an anti-BCMA/anti-CD3 antibody) and a pharmaceutically acceptable carrier. In certain embodiments, the disclosed pharmaceutical compositions are formulated for administration by injection, such as intravenous injection.

Various delivery systems are known and can be used to administer the pharmaceutical compositions of the present disclosure, e.g., encapsulation in liposomes, microparticles, microcapsules, recombinant cells capable of expressing the mutant viruses, receptor mediated endocytosis (see, e.g., Wu et al., 1987, J. Biol. Chem. 262: 4429-4432). Methods of administration include, but are not limited to, intradermal, intramuscular, intraperitoneal, intravenous, subcutaneous, intranasal, epidural, and oral routes. The compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. In some embodiments, the IL-4/IL-13 pathway inhibitor and/or the plasma cell ablating agent is administered intravenously. In some embodiments, the IL-4/IL-13 pathway inhibitor and/or the plasma cell ablating agent is administered subcutaneously.

In some embodiments, a pharmaceutical composition of the present disclosure is contained within a container. Thus, in another aspect, containers comprising a pharmaceutical composition as disclosed herein are provided. For example, in some embodiments, a pharmaceutical composition is contained within a container selected from the group consisting of a glass vial, a syringe, a pen delivery device, and an autoinjector.

In some embodiments, a pharmaceutical composition of the present disclosure is delivered subcutaneously or intravenously with a standard needle and syringe. In some embodiments, the syringe is a pre-filled syringe. In addition, with respect to subcutaneous delivery, a pen delivery device or autoinjector readily has applications in delivering a pharmaceutical composition of the present disclosure. A pen delivery device can be reusable or disposable. Typically, a reusable pen delivery device utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Examples of suitable pen and autoinjector delivery devices include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, IN), NOVOPEN™ 1, ∥ and Ill (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™ OPTIPEN STARLET™, and OPTICLIK™ (sanofi-aventis, Frankfurt, Germany). Examples of disposable pen delivery devices having applications in subcutaneous delivery of a pharmaceutical composition of the present disclosure include, but are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L. P.), and the HUMIRA™ Pen (Abbott Labs, Abbott Park IL).

In certain situations, one or both pharmaceutical compositions can be delivered in a controlled release system. In one embodiment, a pump may be used. In another embodiment, polymeric materials can be used. See, Medical Applications of Controlled Release, Langer and Wise (eds.), 1974, CRC Pres., Boca Raton, Fla. In yet another embodiment, a controlled release system can be placed in proximity of the composition's target, thus requiring only a fraction of the systemic dose (see, e.g., Goodson, 1984, MEDICAL APPLICATIONS OF CONTROLLED RELEASE, vol. 2, pp. 115-138). Other controlled release systems are discussed in Langer, 1990, Science 249:1527-1533.

Suitable injectable preparations may include dosage forms for intravenous, subcutaneous, intracutaneous and intramuscular injections, drip infusions, etc. These injectable preparations may be prepared by known methods. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

In some embodiments, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), cartridges, suppositories, etc.

Injectable formulations of the pharmaceutical compositions may be prepared by known methods. For example, the injectable formulation may be prepared, e.g., by dissolving, suspending or emulsifying the inhibitor (e.g., an anti-IL-4R antibody) or a salt thereof in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injectable formulation thus prepared is preferably filled in an appropriate injection ampoule. In some embodiments, an injectable formulation may include a concentration of the inhibitor (e.g., an anti-IL-4R antibody) and one or more pharmaceutically acceptable solvents (e.g., distilled water, saline, etc.).

Exemplary pharmaceutical compositions comprising an anti-IL-4R antibody that can be used in the context of the present disclosure are disclosed, e.g., in U.S. Pat. No. 8,945,559, the portions of which that identify pharmaceutical compositions comprising an anti-IL-4R antibody are hereby incorporated by reference.

### Kits

In certain embodiments, the present disclosure provides for pharmaceutical combinations and kits comprising an IL-4/IL-13 pathway inhibitor as disclosed herein and a plasma cell ablating agent as disclosed herein. In some embodiments, the combination or kit comprises an anti-IL-4R antibody as disclosed herein (e.g., an anti-IL-4R antibody or antigen-binding fragment thereof comprising a HCDR1, HCDR2, and HCDR3 comprising the amino acid sequences of SEQ ID NOs:3, 4, and 5, respectively, and a LCDR1, LCDR2, and LCDR3 comprising the amino acid sequences of SEQ ID NOs:6, 7, and 8, respectively) and a plasma cell ablating agent as disclosed herein (e.g., an anti-BCMA/anti-CD3 bispecific antibody, comprising a first antigen binding domain that binds specifically to BCMA; and a second antigen-binding domain that binds specifically to CD3, wherein the first antigen-binding domain comprises a HCDR1, HCDR2, and HCDR3 comprising the amino acid sequences of SEQ ID NOs:14, 16, and 18, respectively, and a LCDR1, LCDR2, and LCDR3 comprising the amino acid sequences of SEQ ID NOs:22, 24, and 26, respectively; and wherein the second antigen-binding domain comprises a HCDR1 comprising the amino acid sequence of SEQ ID NO: 30 or 38, a HCDR2 comprising the amino acid sequence of SEQ ID NO: 32 or 40, a HCDR3 comprising the amino acid sequence of SEQ ID NO: 34 or 42, and a LCDR1, LCDR2, and LCDR3 comprising the amino acid sequences of SEQ ID NOs:22, 24, and 26, respectively.

In some embodiments, the combination or kit comprising an IL-4/IL-13 pathway inhibitor and a plasma cell ablating agent is for use in a method as disclosed herein. In some embodiments, the combination or kit is for use in treating an allergy or allergic disorder or for reducing or eliminating allergen-specific serum IgE in a subject. In some embodiments, the combination or kit further comprises one or more additional therapeutic agents as disclosed herein.

In some embodiments, the combination or kit comprising an IL-4/IL-13 pathway inhibitor and a plasma cell ablating agent is for use in increasing the efficacy and/or tolerability of an immunotherapy regimen in a subject having an allergy. Thus, in some embodiments, the combination or kit further comprises one or more reagents for an immunotherapy regimen.

In some embodiments, a kit for use as disclosed herein further comprises instructions for use. In some embodiments, a kit for use as disclosed herein comprises one or more containers comprising the IL-4/IL-13 pathway inhibitor and the plasma cell ablating agent. In some embodiments, the kit comprises a first container comprising the IL-4/IL-13 pathway inhibitor and a second container comprising the plasma cell ablating agent.

### Administration Regimens

In some embodiments, the disclosed methods include sequentially administering a therapeutically effective amount of an IL-4/IL-13 pathway inhibitor in combination with a therapeutically effective amount of a plasma cell ablating agent to a subject in need thereof, wherein each therapeutic agent is administered to the subject in one or more doses, e.g., as part of a specific therapeutic dosing regimen. In certain embodiments, the methods of the present disclosure comprise administering the inhibitors for additive or synergistic activity to treat an allergic disease or disorder, a mast cell activation disorder or mastocytosis.

As used herein, “sequentially administering” means that each dose of inhibitor is administered to the subject at a different point in time, e.g., on different days separated by a predetermined interval (e.g., hours, days, weeks or months). In some embodiments, the disclosed methods include sequentially administering to the subject a single initial dose of an IL-4/IL-13 pathway inhibitor, followed by one or more subsequent doses of the IL-4/IL-13 pathway inhibitor. In certain embodiments, the methods further comprise sequentially administering to the subject a single initial dose of a plasma cell ablating agent, followed by one or more subsequent doses of the plasma cell ablating agent.

In some embodiments, the therapeutic dosing regimen comprises administering one or more doses of an IL-4/IL-13 pathway inhibitor in combination with one or more doses of a plasma cell ablating agent. In certain embodiments, the one or more doses of an IL-4/IL-13 pathway inhibitor and/or the one or more doses of a plasma cell ablating agent are administered to the subject at a frequency of about once a day, once every two days, once every three days, once every four days, once every five days, once every six days, once a week, once every two weeks, once every three weeks, once every four weeks, once a month, once every two months, once every three months, once every four months, or less frequently.

As used herein, the expression “in combination with” means that the IL-4/IL-13 pathway inhibitor is administered before, after, or concurrent with the plasma cell ablating agent. The term “in combination with” also includes sequential or concomitant administration of an IL-4/IL-13 pathway inhibitor and a plasma cell ablating agent.

For example, when the IL-4/IL-13 pathway inhibitor is administered “before” the plasma cell ablating agent, the IL-4/IL-13 pathway inhibitor may be administered more than 150 hours, about 150 hours, about 100 hours, about 72 hours, about 60 hours, about 48 hours, about 36 hours, about 24 hours, about 12 hours, about 10 hours, about 8 hours, about 6 hours, about 4 hours, about 2 hours, about 1 hour, about 30 minutes, or about 15 minutes prior to the administration of the plasma cell ablating agent. When the IL-4/IL-13 pathway inhibitor is administered “after” the plasma cell ablating agent, the IL-4/IL-13 pathway inhibitor may be administered about 15 minutes, about 30 minutes, about 1 hour, about 2 hours, about 4 hours, about 6 hours, about 8 hours, about 10 hours, about 12 hours, about 24 hours, about 36 hours, about 48 hours, about 60 hours, about 72 hours, or more than 72 hours after the administration of the plasma cell ablating agent. Administration of the IL-4/IL-13 pathway inhibitor “concurrent” with the plasma cell ablating agent means that the IL-4/IL-13 pathway inhibitor is administered to the subject in a separate dosage form within 10 minutes (before, after, or at the same time) of administration of the plasma cell ablating agent, or administered to the subject as a single combined dosage formulation comprising both the IL-4/IL-13 pathway inhibitor and the plasma cell ablating agent.

As used herein, an “initial dose” is a dose that is administered at the beginning of the treatment regimen (also referred to as a “baseline dose”). The one or more subsequent doses administered after the initial dose may all contain the same amount of the IL-4/IL-13 pathway inhibitor or the plasma cell ablating agent. In certain embodiments, however, the amount contained in the initial, and subsequent doses varies from one another (e.g., adjusted up or down as appropriate) during the course of treatment. In certain embodiments, one or more (e.g., 1, 2, 3, 4, or 5) doses are administered at the beginning of the treatment regimen as “loading doses” followed by subsequent doses that are administered on a less frequent basis (e.g., “maintenance doses”). For example, an IL-4/IL-13 pathway inhibitor or plasma cell ablating agent may be administered to a patient with an allergic disease at a loading dose of about 1 mg/kg to about 20 mg/kg followed by one or more maintenance doses of about 0.1 mg/kg to about 10 mg/kg of the patient's body weight.

In one exemplary embodiment of the present disclosure, each subsequent dose is administered % to 14 weeks or more (e.g., %, 1, 1%, 2, 2%, 3, 3%, 4, 5, 5%, 6, 6%, 7, 7%, 8, 8%, 9, 9%, 10, 10% or more weeks) after the immediately preceding dose. The phrase “the immediately preceding dose,” as used herein, means, in a sequence of multiple administrations, the dose of each inhibitor administered to a subject prior to administration of the next dose in the sequence with no intervening doses.

### Dosage

In certain embodiments, at least one dose of the IL-4/IL-13 pathway inhibitor comprises about 0.1-50 mg/kg, such as about 0.1-10 mg/kg, of the subject's body weight. For example, at least one dose may comprise about 0.1, 1, 0.3, 3, 4, 5, 6, 7, 8, 9 or 10 mg/kg of the subject's body weight. In certain embodiments, at least one dose of the IL-4/IL-13 pathway inhibitor comprises about 0.05-600 mg of the IL-4/IL-13 pathway inhibitor, e.g., about 5-600 mg, about 10-300 mg, about 50-600 mg, or about 50-300 mg, such as about 5, 10, 15, 20, 25, 40, 45, 50, 60, 70, 80, 90, 100, 200, 300, 400 mg, 500 mg, 600 mg or more of the IL-4/IL-13 pathway inhibitor. In one embodiment, the IL-4/IL-13 pathway inhibitor is REGN668 (dupilumab).

In certain embodiments, at least one dose of the plasma cell ablating agent comprises about 0.1-20 mg/kg of the subject's body weight, such as about 0.1, 1, 0.3, 3, 4, 5, 6, 7, 8, 9 or 10 mg/kg of the subject's body weight. In certain embodiments, at least one dose of the plasma cell ablating agent comprises about 0.05-500 mg of the plasma cell ablating agent, such as about 5, 10, 15, 20, 25, 40, 45, 50, 60, 70, 80, 90, 100 mg or more of the plasma cell ablating agent. In one embodiment, the plasma cell ablating agent is an anti-BCMA/anti-CD3 bispecific antibody (such as REGN5459). In one embodiment, the plasma cell ablating agent is a proteasome inhibitor such as bortezomib.

The amounts of IL-4/IL-13 pathway inhibitor and plasma cell ablating agent administered to a subject according to the methods disclosed herein is a therapeutically effective amount. As used herein, the term “therapeutically effective amount” means an amount of each therapeutic agent that results in one or more of: (a) a reduction in the severity or duration of a symptom or an indication of an allergy—e.g., anaphylaxis; (b) reduction in the level of serum allergen-specific IgE; (c) elimination of serum IgE in the subject; (d) reduction of allergen sensitization; (e) reduction in susceptibility to an allergic reaction and/or (f) a reduction in the use or need for conventional anti-allergy therapy (e.g., reduced or eliminated use of corticosteroids) as compared to an untreated subject or a subject treated with either therapeutic agent as monotherapy.

In the case of an IL-4/IL-13 pathway inhibitor (e.g., anti-IL-4R antibody), a therapeutically effective amount can be from about 0.05 mg to about 600 mg, e.g., about 0.05 mg, about 0.1 mg, about 1.0 mg, about 1.5 mg, about 2.0 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, or about 600 mg, of the IL-4/IL-13 pathway inhibitor. In certain embodiments, 10 mg, 25 mg, 50 mg, 75 mg, 150 mg, or 300 mg of an IL-4/IL-13 pathway inhibitor is administered to a subject.

In the case of a plasma cell ablating agent (e.g., an anti-BCMA/anti-CD3 bispecific antibody), a therapeutically effective amount can be from about 0.05 mg to about 500 mg, from about 1 mg to about 500 mg, from about 10 mg to about 450 mg, from about 50 mg to about 400 mg, from about 75 mg to about 350 mg, or from about 100 mg to about 300 mg of the antibody. For example, in various embodiments, the amount of the plasma cell ablating agent is about 0.05 mg, about 0.1 mg, about 1.0 mg, about 1.5 mg, about 2.0 mg, about 5 mg, about 10 mg, about 15 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, or about 500 mg, of the plasma cell ablating agent.

In certain embodiments, an individual dose amount of an IL-4/IL-13 pathway inhibitor (e.g., an anti-IL-4R antibody) and/or a plasma cell ablating agent (e.g., an anti-BCMA/anti-CD3 antibody) administered to a subject may be less than a therapeutically effective amount, i.e., a subtherapeutic dose. For example, if the therapeutically effective amount of an inhibitor comprises 3 mg/kg, a subtherapeutic dose comprises an amount less than 3 mg/kg, e.g., 2 mg/kg, 1.5 mg/kg, 1 mg/kg, 0.5 mg/kg or 0.3 mg/kg. As defined herein, a “subtherapeutic dose” refers to an amount of the inhibitor that does not lead to a therapeutic effect by itself. However, in certain embodiments, multiple subtherapeutic doses of the inhibitor may be administered to collectively achieve a therapeutic effect in the subject.

### EXAMPLES

The disclosed technology is next described by means of the following examples. The use of these and other examples anywhere in the specification is illustrative only, and in no way limits the scope and meaning of the disclosure or of any exemplified form. Likewise, the disclosure is not limited to any particular preferred embodiments described herein. Indeed, modifications and variations of the disclosure may be apparent to those skilled in the art upon reading this specification and can be made without departing from its spirit and scope. The disclosure is therefore to be limited only by the terms of the claims, along with the full scope of equivalents to which the claims are entitled. Also, while efforts have been made to ensure accuracy with respect to numbers used (e.g., amounts, temperature, etc.), some experimental errors and deviations should be accounted for. Unless indicated otherwise, parts are parts by weight, molecular weight is average molecular weight, temperature is in degrees Centigrade, and pressure is at or near atmospheric.

### Example 1: Effect of an Anti-IL-4R Antibody in Combination with an Anti-BCMA/Anti-CD3 Bispecific Antibody

This example relates to a study that demonstrates the enhanced efficacy of an IL-4/IL-13 pathway inhibitor in combination with a plasma cell ablating agent in blocking IgE production in a mouse model of chronic allergen-driven lung inflammation.

The IL-4/IL-13 pathway inhibitor used in this example was a mouse anti-IL-4R antibody identified as REGN1103, which is a mouse surrogate antibody of a human monoclonal antibody identified as REGN668 (also known as dupilumab) directed to human IL-4R. REGN1103 comprises a HCVR/LCVR amino acid sequence pair of SEQ ID NOs: 43/44 and has an affinity for mouse IL-4R that is in a similar range as the affinity of dupilumab for human IL-4R. In addition, REGN1103 inhibits IL-4- and IL-13-dependent proliferation of cell lines at IC50s of 1.9 nM and 11 pM, respectively.

The plasma cell ablating agent used in this example was an anti-BCMA/anti-CD3 bispecific antibody REGN5459, that comprises an anti-BCMA binding domain comprising a HCVR of SEQ ID NO: 12 and a LCVR of SEQ ID NO: 20; and an anti-CD3 binding domain comprising a HCVR of SEQ ID NO: 36 and a LCVR of SEQ ID NO: 20.

### Materials and Methods

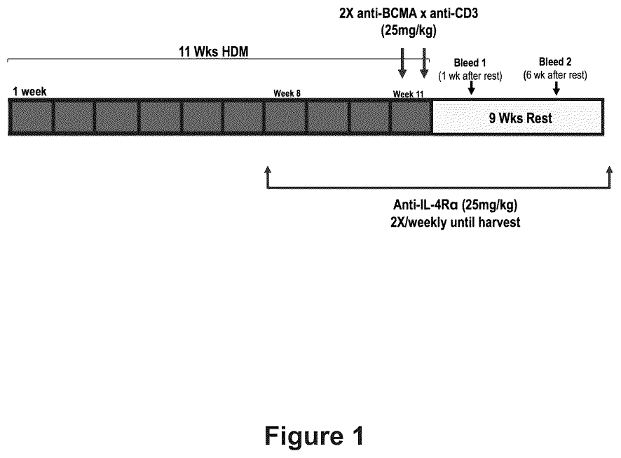

To determine the effect of combining anti-IL-4Rα and anti-BCMA×anti-CD3 on IgE production in a relevant in vivo model, a chronic house dust mite (HDM) driven lung inflammation study was conducted in mice that were homozygous for human BCMA and human CD3 in place of mouse BCMA and CD3. Chronic lung inflammation and persistent IgE production was induced by exposing mice to 25 μg of HDM (Greer, Catalog #XPB70D3A25) diluted in 20 μL of saline (Sigma, Catalog #S8776) or 20 μL of saline (control group) intranasally (i.n.) three times a week for 11 weeks. This model induces class switching of B cells into IgE producing plasma cells in secondary lymphoid organs and drives accumulation of IgE plasma cells in the bone marrow. At week 8 after the first HDM administration, a subset of mice started receiving subcutaneous injections of 25 mg/kg REGN1103 (anti-IL-4R %) or 25 mg/kg isotype control until the end of the experiment. At week 11, two doses of REGN5459 (anti-BCMA×anti-CD3) or two doses of an isotype control antibody were administered subcutaneously and mice were rested in the absence of i.n. HDM for 9 weeks. Details of HDM exposure and antibody treatment protocol is outlined in Table 1 and FIG. 1.

TABLE 1
HDM exposure and antibody treatment protocol for mice

| | BCMA × CD3 | Intranasal | | Antibody | Antibody |
|---|---|---|---|---|---|
| | humanized | (i.n.) | | (2× weekly | (2 doses at |
| Group | mice | (11 weeks) | Rest | after week 8) | week 11) |
| A | 8 | Saline | 9 wks | None | None |
| B | 9 | HDM | 9 wks | None | None |
| C | 10 | HDM | 9 wks | Isotype | Isotype |
| | | | | control | control |
| D | 8 | HDM | 9 wks | Isotype | REGN5459 |
| | | | | control | (anti-BCMA × |
| | | | | | anti-CD3) |
| E | 9 | HDM | 9 wks | REGN1103 | Isotype |
| | | | | (anti-IL- | control |
| | | | | 4Rα) | |
| F | 9 | HDM | 9 wks | REGN1103 | REGN5459 |
| | | | | (anti-IL- | (anti-BCMA × |
| | | | | 4Rα) | anti-CD3) |

1 week and 6 weeks after the last dose of HDM, ˜100 μL of blood was collected from all groups of mice by retro-orbital bleeding and transferred into microtainer tubes (BD, Catalog #365967) for serum isolation. The total IgE concentration in serum was determined using an OptEIA™ ELISA kit (BD Biosciences, #555248) following manufacturer's instructions. Briefly, ELISA plates were coated with IgE capture antibody diluted in coating buffer (carbonate-bicarbonate buffer, Sigma; catalog #C3041, diluted in 100 mL distilled water, Gibco; catalog #15230-270) overnight at 4°. Coated plates were washed 4× in wash buffer (0.05% Tween 20, Sigma; catalog #P1379 diluted in DPBS, GE; catalog #SH3001304). Plates were blocked with Assay Diluent (BD; catalog #555213) for 1 hour at room temperature (RT). 100 μL of serum samples diluted at a starting concentration of 1:50 or 1:100 with further 3-fold serial dilutions and IgE standard diluted at a starting concentration of 100 ng/mL with further 2-fold serial dilution was added to plates and incubated for 2 hrs at RT. Plates were then washed 4× in wash buffer and incubated in 100 μL working detector (detection antibody with sAv-HRP) for 1 hr at RT. Plates were washed 7×, soaked in wash buffer for 5 m and washed again 4× to remove unbound detection antibody. 100 μL of TMB Substrate Solution (ED; catalog #555214) was added to each sample and plates were incubated in the dark for 30 min followed by addition of 50 μL Stop Solution (2N Sulfuric Acid, BDH VWR analytical; catalog #BDH7500). Absorbance was measured at 450 nm and IgE concentrations were calculated from a standard curve. Serum IgE values are shown as ng/mL Statistical significance was determined by Kruskal-Wallis test with Dunn's post-hoc multi-comparison test in GraphPad Prism.

### Results

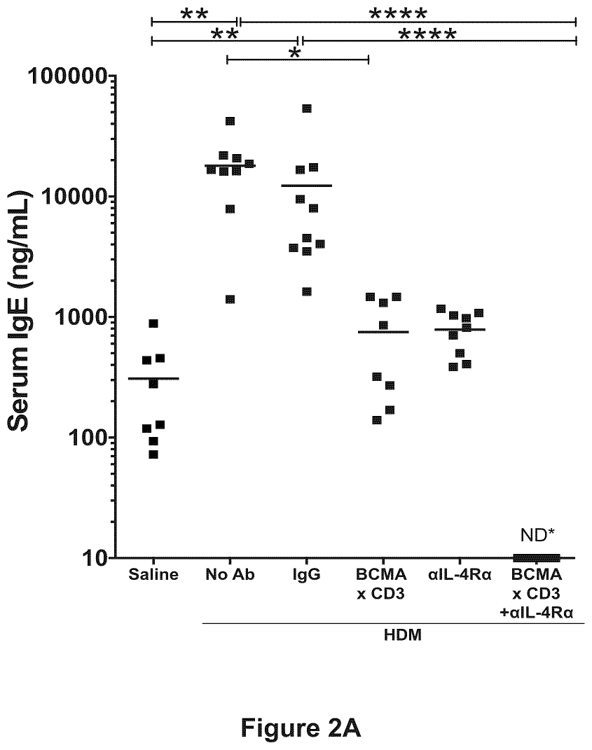

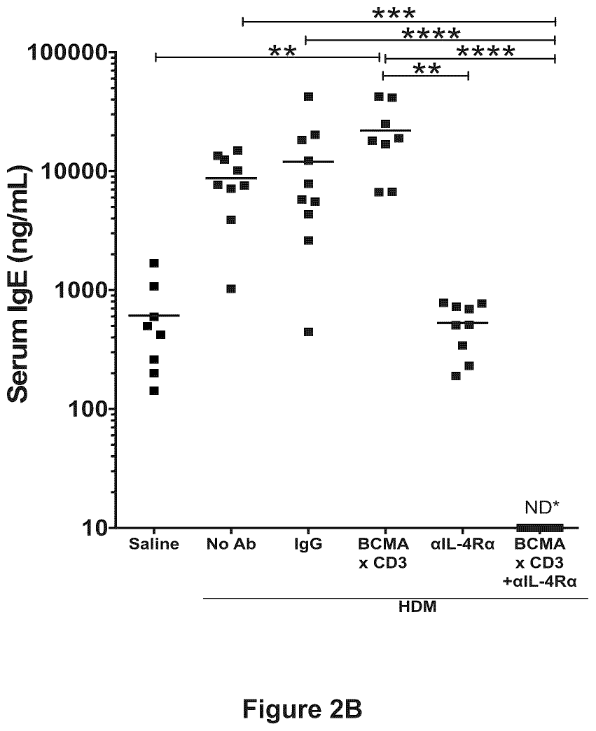

In a chronic HDM model, anti-IL4Ra treatment by itself reduced, but did not eliminate, circulating IgE (FIGS. 2A and 21B; Table 2).

TABLE 2
Effect of anti-IL-4Rα and anti-BCMA × anti-CD3 antibody
treatments on serum IgE in a chronic HDM model

| | | Mean rank | | Mean rank |
|---|---|---|---|---|
| | Mean ± SD | difference | Mean ± SD | difference |
| | serum IgE | compared to | serum IgE | compared to |
| | (ng/mL) | Isotype control | in (ng/mL) | Isotype control |
| Group | 1 wk rest | 1 wk rest | 6 wks rest | 6 wks rest |
| A | 308.5 ± 277.4 | −25.58 (**) | 610.8 ± 524.9 | −18.85 |
| (Saline) | (n = 8) | | (n = 8) | |
| B | 17981 ± 11162 | 3.578 | 8725 ± 4545 | 0.2333 |
| (HDM) | (n = 9) | | (n = 9) | |
| C | 12279 ± 15582 | N/A | 11997 ± 12524 | N/A |
| (Isotype control) | (n = 10) | | (n = 10) | |
| D | 750.5 ± 596.1 | −18.45 | 21995 ± 13734 | 8.025 |
| (anti-BCMA × | (n = 8) | | (n = 8) | |
| anti-CD3) | | | | |
| E | 786.1 ± 300.4 | −16.76 | 528.5 ± 231.6 | −17.99 |
| (anti-IL4Rα) | (n = 9) | | (n = 9) | |
| F | 0 ± 0 | −37.2 (****) | 0 ± 0 | −32.1 (****) |
| (anti-BCMA × | (n = 9) | | (n = 9) | |
| anti-CD3 + | | | | |
| anti-IL-4Rα) | | | | |

Anti-BCMA×anti-CD3 treatment alone transiently reduced IgE production, but serum IgE levels were restored after 6 weeks of rest (FIGS. 2A & 21B; Table 2). The combination of anti-IL-4Rα with anti-BCMA×anti-CD3 treatment eliminated serum IgE (undetectable by IgE ELISA) (FIGS. 2A & 2B; Table 2) demonstrating the efficacy of blocking IL-4Rα and depleting plasma cells with anti-BCMA×anti-CD3 as a successful strategy to block IgE production.

### Example 2: Therapeutic Combination of a BCMAxCD3 Bispecific Antibody with an Anti-IL-4Rα Antibody Completely Blocks HDM-Specific IgE Production During Ongoing HDM Exposure

This example demonstrates the efficacy of blocking IL-4R and depleting plasma cells in blocking allergen-specific IgE production even in the presence of continuous allergen exposure.

In this example, the IL-4/IL-13 pathway inhibitor that was used was the mouse anti-IL-4R antibody REGN1103, which is a mouse surrogate antibody of a human monoclonal antibody identified as REGN668 (also known as dupilumab) directed to human IL-4R. REGN1103 is described in Example 1 above. The plasma cell ablating agent that was used was the anti-BCMA×anti-CD3 bispecific antibody REGN5459, described in Example 1 above. A mouse IgG1 antibody (REGN1094) and a human IgG4×anti-CD3 antibody (REGN4460) were used as isotype controls.

### Materials and Methods

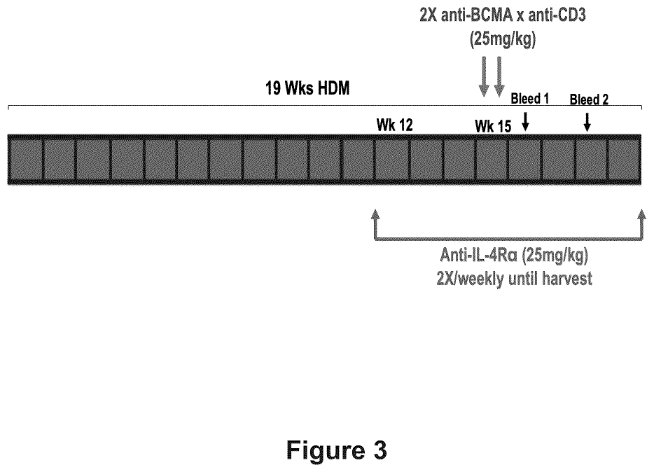

To determine the effect of combining anti-IL-4Rα and anti-BCMA×anti-CD3 on IgE production in a relevant in vivo model, a chronic house dust mite (HDM) driven lung inflammation study was conducted in mice that were homozygous for human BCMA and human CD3 in place of mouse BCMA and CD3. Chronic lung inflammation and persistent IgE production was induced by exposing mice to 25 μg of HDM (Greer, Catalog #XPB70D3A25) diluted in 20 μL of saline (Sigma, Catalog #S8776) or 20 μL of saline (control group) intranasally (i.n.) three times a week for 19 weeks. This model induces class switching of B cells into IgE producing plasma cells in secondary lymphoid organs and drives accumulation of IgE plasma cells in the bone marrow. At week 12 after the first HDM administration, a subset of mice started receiving subcutaneous injections of 25 mg/kg REGN1103 (anti-IL-4R %) or 25 mg/kg REGN1094 (isotype control) until the end of the experiment. At week 15, two doses of REGN5459 (anti-BCMA×anti-CD3) or two doses of REGN4460 (isotype control) were administered subcutaneously and mice were exposed to HDM for an additional 4 weeks. Details of HDM exposure and antibody treatment protocol are shown in Table 3 below and in FIG. 3.

TABLE 3
HDM exposure and antibody treatment protocol
for mice in a chronic (19 weeks) HDM model

| | BCMA × CD3 | Intranasal | Antibody | Antibody |
|---|---|---|---|---|
| | humanized | (i.n.) | (2× weekly | (2 doses at |
| Group | mice | (11 weeks) | after week 8) | week 11) |
| A | 9 | Saline | None | None |
| B | 11 | HDM | None | None |
| C | 10 | HDM | REGN1094 | REGN4460 |
| | | | (Isotype | (Isotype |
| | | | control) | control) |
| D | 9 | HDM | REGN1094 | REGN5459 |
| | | | (Isotype | (anti-BCMA × |
| | | | control) | anti-CD3) |
| E | 11 | HDM | REGN1103 | REGN4460 |
| | | | (anti-IL-4Rα) | (Isotype |
| | | | | control) |
| F | 10 | HDM | REGN1103 | REGN5459 |
| | | | (anti-IL-4Rα) | (anti-BCMA × |
| | | | | anti-CD3) |

1 week, 3 weeks and 5 weeks after REGN5459 administration, ˜100 μL of blood was collected from all groups of mice by retro-orbital bleeding and transferred into microtainer tubes (BD, Catalog #365967) for serum isolation. The HDM-specific IgE concentration in serum was determined using a Mouse Serum Anti-HDM IgE Antibody Assay kit (Chondrex catalog #3037) following manufacturer's instructions. Briefly, 100 μL of serum samples diluted at a concentration of 1:20 or 1:60, and HDM-IgE standard diluted at a starting concentration of 50 ng/mL with further 2-fold serial dilution was added to pre-coated plates provided with the kit and incubated at 4° C. overnight. Plates were then washed 3× in wash buffer and incubated in 100 μL of biotinylated HDM, provided with the kit. Plates were then washed 4× in wash buffer and incubated in 100 μL streptavidin peroxidase (provided with the kit) for 30 min at RT. Plates were washed 7× and 100 μL of TMB Substrate Solution (provided with the kit) was added to each sample and plates were incubated in the dark for 25 min followed by addition of 50 μL Stop Solution (2N Sulfuric Acid, provided with the kit). Absorbance was measured at 450 nm and HDM-IgE concentrations were calculated from a standard curve. Serum IgE values are shown as ng/mL. Lower Limit of Quantification (LLOQ) for HDM-Specific IgE ELISA was 15.62 ng/mL. Statistical significance was determined by Kruskal-Wallis test with Dunn's post-hoc multi-comparison test in GraphPad Prism.

### Results

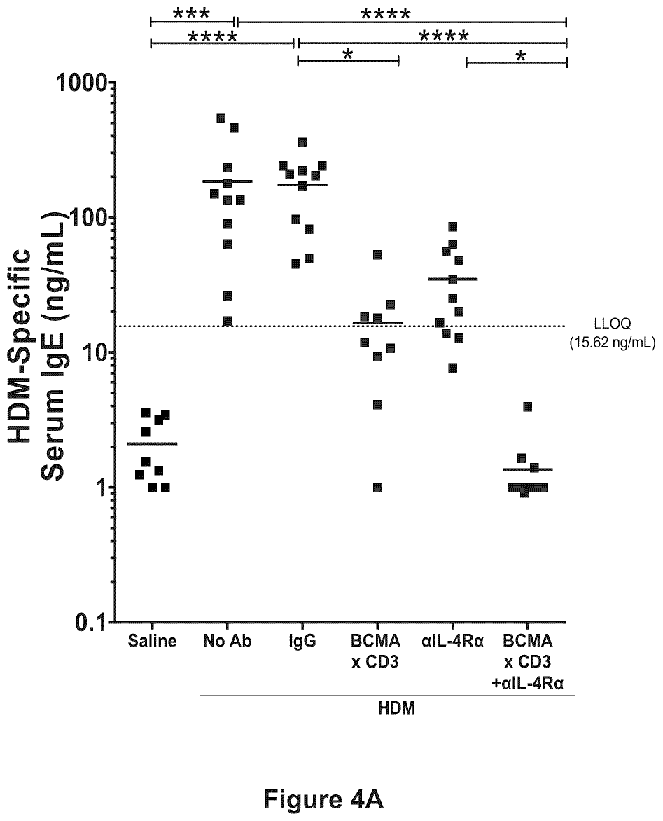

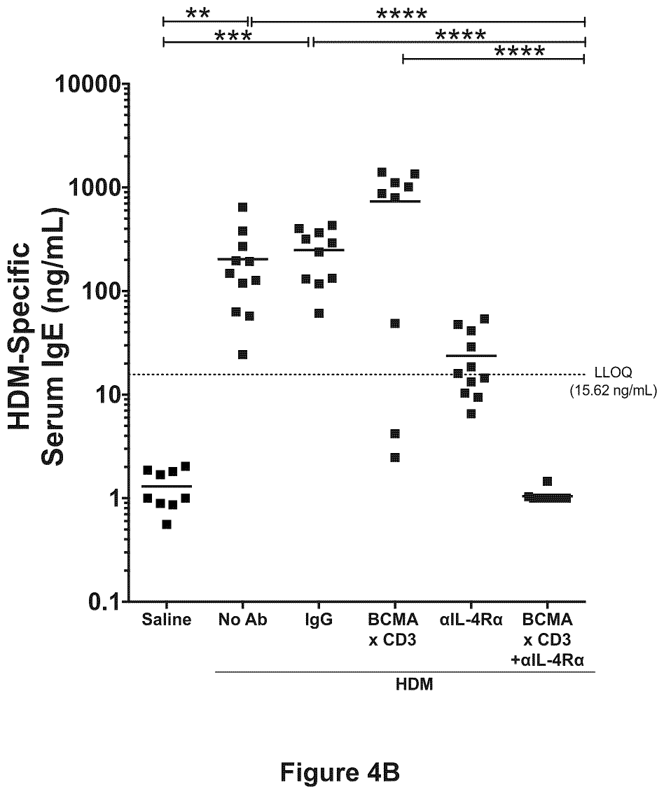

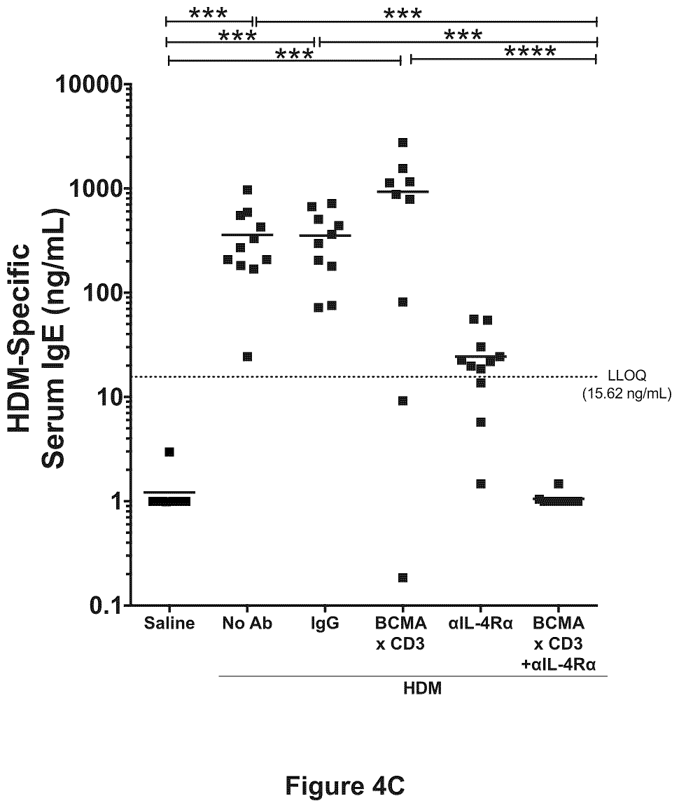

In a chronic HDM model, anti-IL4Ra treatment showed a trend towards reduced serum HDM-specific IgE levels that did not reach statistical significance, and this trend was maintained across the three time points examined (see, FIGS. 4A, 4B, and 4C and Table 4). Anti-BCMA×anti-CD3 treatment alone led to a significant reduction in circulating HDM-specific IgE relative to the isotype control group one week after REGN5459 administration (FIG. 4A), but this effect was short-lived and at 3 weeks or 5 weeks after bispecific administration the levels of HDM-specific IgE were not significantly different relative to the isotype control group (FIGS. 41 and 40 and Table 4). The combination of anti-IL-4R with anti-BCMA×anti-CD3 treatment eliminated serum HDM-specific IgE (undetectable by ELISA). This effect was maintained for the duration of the experiment (see, FIGS. 4A, 41B, and 40 and Table 4), demonstrating the efficacy of blocking IL-4Rα and depleting plasma cells with anti-BCMA×anti-CD3 as a successful strategy to block allergen-specific IgE production even in the presence of continuous allergen exposure.

TABLE 4
HDM-specific IgE at 1, 3, and 5 weeks post anti-BCMAxCD treatment

| | | Mean rank | | Mean rank | | |
|---|---|---|---|---|---|---|
| | | difference | | difference | | Mean rank |
| | | compared to | Mean ± SD | compared to | Mean ± SD | difference |
| | Mean ± SD | Isotype | serum IgE | Isotype | serum IgE | compared to |
| | serum IgE | control | in (ng/mL) | control | in (ng/mL) | Isotype control |
| | (ng/mL) | 1 wk post | 3 wk post | 3 wk post | 5 wk post | 5 wk post |
| Group | 1 wk rest | BCMA × CD3 | BCMA × CD3 | BCMA × CD3 | BCMA × CD3 | BCMA × CD3 |
| A | Below LLOQ | −37.47 (****) | Below LLOQ | −32.61 (***) | Below LLOQ | −32.9 (***) |
| (Saline) | (n = 9) | | (n = 9) | | (n = 9) | |
| B | 185.0 ± 170.7 | −2.273 | 202.3 ± 179.4 | −3.318 | 357.9 ± 264.9 | −0.2636 |
| (HDM) | (n = 11) | | (n = 11) | | (n = 11) | |
| C | 175.0 ± 97.56 | N/A | 248.7 ± 131.8 | N/A | 352.4 ± 229.5 | N/A |
| (Isotype control) | (n = 11) | | (n = 10) | | (n = 10) | |
| D | 16.49 ± 15.50 | −24.64 (*) | 733.9 ± 571.3 | 1.056 | 930.3 ± 887.6 | 2.433 |
| (anti-BCMA × | (n = 9) | | (n = 9) | | (n = 9) | |
| anti-CD3) | | | | | | |
| E | 34.90 ± 25.12 | −16.64 | 23.67 ± 16.67 | −18.05 | 24.4 ± 17.23 | −16.85 |
| (anti-IL-4Rα) | (n = 11) | | (n = 11) | | (n = 11) | |
| F | Below LLOQ | −42.23 (****) | Below LLOQ | −38.10 (****) | Below LLOQ | −34.15 (***) |
| (anti-BCMA × | (n = 11) | | (n = 10) | | (n = 10) | |
| anti-CD3 + | | | | | | |
| anti-IL-4Rα) | | | | | | |

### Example 3: Effect of Combination Treatment with a BCMAxCD3 Bispecific Antibody and an Anti-IL-4Rα Antibody on IgE Bone Marrow Plasma Cells

The mice described in Example 2 were also analyzed for IgE bone marrow plasma cells. Following the HDM exposure and antibody treatment protocol as described in Table 3 and FIG. 3, the mice were sacrificed, and femurs were collected from the mice. Bone marrow was extracted from the femurs by cutting both ends of each bone, placing each bone in an individual well of a 96-well PCR plate with holes cut in the bottom of each well, then placing the PCR plate on top of a 96-well 2 mL deep well collection plate and centrifuging the plate for 4 minutes at 500 g. The bone marrow was resuspended in 0.5 mL of RBC lysis buffer and incubated for 3 minutes at room temperature, followed by addition of 1-2 mL of PBS to deactivate the lysis buffer. Cells were centrifuged at 400 g for 4 minutes, the supernatant decanted and the pellet resuspended in 1 ml DPBS and filtered through Millipore plate filter (100 μm) into a 2 mL deep well plate. The cells were then centrifuged and resuspended in in 200 μL of PBS. The bone marrow cells were then plated in 96-well plates and stained with a live/dead cell marker, followed by antibody staining with the antibodies B220, CD138, IgM, IgG1, IgA, IgD, IgE (extracellular block), and “Dump” (including TCRβ, CD200R3, Ly6G, CD49b, and CD11b).

After staining, the cells were washed twice with MACS buffer, fixed with BD Cytofix (cat #554655) diluted 1:4 in PBS for 15 minutes, then resuspended in MACS buffer and stored at 4° C. On the day of acquisition, the cells were washed, incubated in BD Perm/wash buffer (cat #554723) for 10 minutes and stained with the intracellular antibodies Light Chain κ, IgG1, and Intra IgE. The cells were then acquired in an LSRFortessa instrument and analyzed using FlowJo software. Mature IgE bone marrow plasma cells were identified as Live, Dump-B220-Light Chain κ+ IgE+. Percent reduction of plasma cells in individual antibody-administered mice was calculated with the following formula: 100−(100×percent plasma cells/mean percent plasma cells in the isotype group), where percent plasma cells are calculated relative to total live cells.

### Results

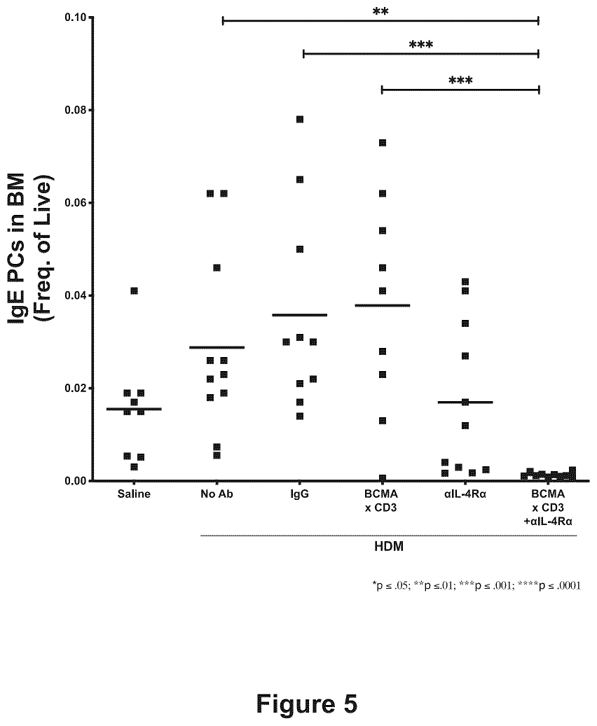

In a chronic HDM model with continuous HDM exposure after antibody treatments, neither anti-IL4Ra or BCMA×CD3 administration alone had a significant impact on IgE bone marrow plasma cells at the time of harvest (5 weeks after BCMA×CD3 administration), although treatment with anti-II-4Ra showed a trend towards reduced IgE bone marrow plasma cells (see FIG. 5 and Table 5). By contrast, the combination of continuous anti-IL-4Rα administration with transient anti-BCMA×anti-CD3 administration led to a significant reduction in IgE bone marrow plasma cells relative to both the untreated and the isotype control (see FIG. 5 and Table 5).

TABLE 5
IgE bone marrow plasma cells (BMPCs)
following antibody treatment

| | | | Mean Percent | |
|---|---|---|---|---|
| | | | Decrease Relative | |
| | | | to Average | |
| | | Plasma cells | Plasma Cells in | |
| | | (percent of | isotype control | |
| | Treatment | Live ± SD) | group | |
| IgE BMPCs | Anti-BCMA × | 0.0378 ± 0.023 | −5.72 | (ns) |
| | Anti CD3 | | | |
| | Anti-IL4Rα | 0.017 ± 0.016 | 48.46 | (ns) |
| | Anti-BCMA × | 0.0013 ± 0.0005 | 96.14 | (***) |
| | Anti CD3 + | | | |
| | Anti-IL4Rα | | | |

### Example 4: Effect of Combination Treatment with a BCMAxCD3 Bispecific Antibody and an Anti-IL-4Rα Antibody on IgE Production and IgE-Producing Cells

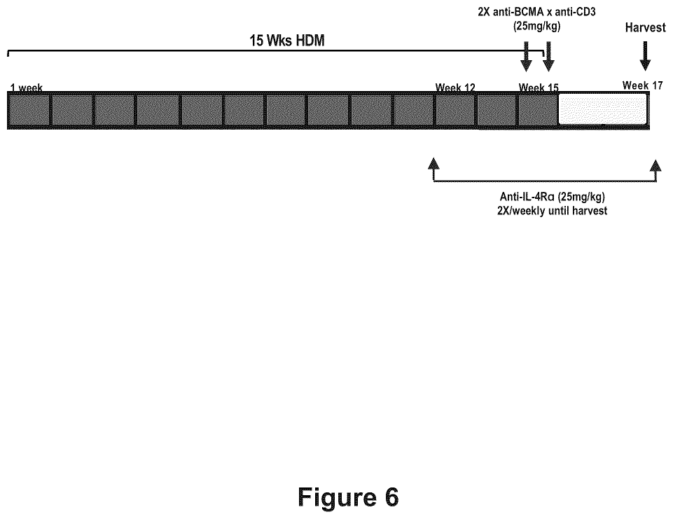

To determine the effect of combination treatment with anti-IL-4Rα and anti-BCMA×anti-CD3 on IgE production and IgE-producing cells in a relevant in vivo model, a HDM-driven lung inflammation study was conducted in mice that were homozygous for human BCMA and human CD3 in place of mouse BCMA and CD3 as described in Examples 1-2 above. Chronic lung inflammation and persistent IgE production was induced by exposing mice to 25 μg of HDM diluted in 20 μL of saline or to 20 μL of saline (control group) intranasally (i.n.) three times a week for 15 weeks. This model induces class switching of B cells into IgE producing plasma cells in secondary lymphoid organs and drives accumulation of IgE plasma cells in the bone marrow. At week 12 after the first HDM administration, a subset of mice started receiving subcutaneous injections of 25 mg/kg REGN1103 (anti-IL-4Ra) or 25 mg/kg REGN1094 (isotype control) until the end of the experiment. At week 15, two doses of REGN5459 (anti-BCMA×anti-CD3) or two doses of REGN4460 (isotype control) were administered subcutaneously and mice were rested for 2 weeks without further HDM administration. Details of HDM exposure and antibody treatment protocol are outlined in FIG. 6 and in Table 6 below.

TABLE 6
HDM exposure and antibody treatment protocol for mice
to address the effect of anti-IL-4Rαand anti-BMCA ×
anti-CD3 antibody treatments on bone marrow plasma
cell populations in a chronic (15 weeks) HDM model

| | BCMA × CD3 | Intranasal | Antibody | Antibody |
|---|---|---|---|---|
| | humanized | (i.n.) | (2× weekly | (2 doses at |
| Group | mice (#) | (15 weeks) | after week 12) | week 15) |
| A | 9 | Saline | None | None |
| B | 11 | HDM | None | None |
| | | (12 weeks) | | |
| C | 13 | HDM | None | None |
| D | 10 | HDM | REGN1094 | REGN4460 |
| | | | (Isotype | (Isotype |
| | | | control) | control) |
| E | 9 | HDM | REGN1094 | REGN5459 |
| | | | (Isotype | (anti-BCMA × |
| | | | control) | anti-CD3) |
| F | 10 | HDM | REGN1103 | REGN4460 |
| | | | (anti-IL-4Rα) | (Isotype |
| | | | | control) |
| G | 10 | HDM | REGN1103 | REGN5459 |
| | | | (anti-IL-4Rα) | (anti-BCMA × |
| | | | | anti-CD3) |

Following the HDM exposure and antibody treatment protocol, the mice were sacrificed, and blood, spleen, and bones were taken. The blood was collected from all groups of mice by cardiac puncture and transferred into microtainer tubes (BD, Catalog #365967) for serum isolation. The HDM-specific IgE concentration in serum was determined using a Mouse Serum Anti-HDM IgE Antibody Assay kit (Chondrex catalog #3037) following manufacturer's instructions. Briefly, 100 μL of serum samples diluted at a concentration of 1:10 or 1:60, and HDM-IgE standard diluted at a starting concentration of 50 ng/mL with further 2-fold serial dilution was added to pre-coated plates provided with the kit and incubated at 4° C. overnight. Plates were then washed 3× in wash buffer and incubated in 100 μL of biotinylated HDM, provided with the kit. Plates were then washed 4× in wash buffer and incubated in 100 μL streptavidin peroxidase (provided with the kit) for 30 min at RT. Plates were washed 7× and 100 μL of TMB Substrate Solution (provided with the kit) was added to each sample and plates were incubated in the dark for 25 min followed by addition of 50 μL Stop Solution (2N Sulfuric Acid, provided with the kit). Absorbance was measured at 450 nm and HDM-IgE concentrations were calculated from a standard curve. Serum IgE values are shown as ng/mL. Statistical significance was determined by Kruskal-Wallis test with Dunn's post-hoc multi-comparison test in GraphPad Prism.

Spleens and femurs were also collected from the mice. Spleens were mashed on a 74-micron cell strainer in 2 ml RPMI media using the back end of a 3 mL syringe, and the single cell suspensions were transferred to a 96-well plate. Bone marrow was extracted from the femurs by cutting both ends of each bone, placing each bone in an individual well of a 96-well PCR plate with holes cut in the bottom of each well, then placing the PCR plate on top of a 96-well deep 2 mL deep well collection plate and centrifuging the plate for 4 minutes at 500 g. Spleen samples were resuspended in 1 mL and bone marrow in 0.5 mL of RBC lysis buffer and incubated for 3 minutes at room temperature, followed by addition of 1-2 mL of PBS to deactivate the lysis buffer. Cells were centrifuged at 400 g for 4 minutes, the supernatant decanted and the pellet resuspended in 1 mL DPBS and filtered through Millipore plate filter (100 μm) into a 2 mL deep well plate. The cells were then centrifuged and the splenic cells resuspended in 1 mL and the bone marrow in 200 μL of PBS. One tenth of the spleen cells and all of the bone marrow cells were then plated in 96-well plates and stained with a live/dead cell marker, followed by antibody staining with the antibodies B220, CD138, IgM, IgG1, IgA, IgD, IgE (extracellular block), and “Dump” (including TCRβ, CD200R3, Ly6G, CD49b, and CD11b). After staining, the cells were washed twice with MACS buffer, fixed with BD Cytofix (cat #554655) diluted 1:4 in PBS for 15 minutes, then resuspended in MACS buffer and stored at 4 degrees. On the day of acquisition, the cells were washed, incubated in BD Perm/wash buffer (cat #554723) for 10 minutes and stained with the intracellular antibodies Light Chain κ, IgG1, and Intra IgE. The cells were then acquired in an LSRFortessa instrument and analyzed using FlowJo software. Mature plasma cells were identified as Live, Dump-(dump includes TCRb, CD200R3, Ly6G, CD49b and CD11b) B220-Light Chain κ+. Percent reduction of plasma cells in individual antibody-administered mice was calculated with the following formula: 100−(100×percent plasma cells/mean percent plasma cells in the isotype group), where percent plasma cells are calculated relative to total live cells. The results are shown in Table 7 below.

### Results

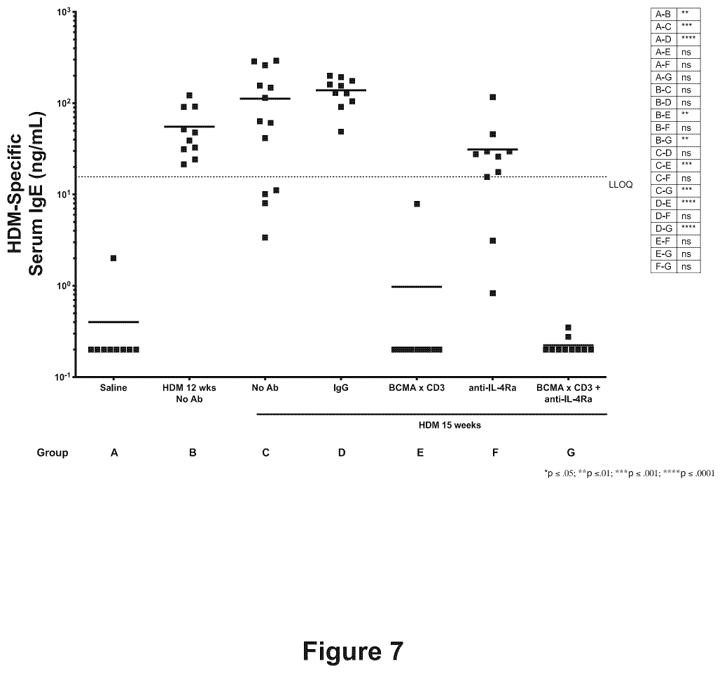

In a chronic HDM model, anti-IL4Ra treatment (Group F) showed a trend towards reduced serum HDM-specific IgE levels with most samples having detectable levels of HDM-specific IgE (see, Table 7 and FIG. 7). Both the anti-BCMA×anti-CD3 treatment alone (Group E) and the combination of anti-IL-4Rα with anti-BCMA×anti-CD3 treatment (Group G) eliminated serum HDM-specific IgE, with all mice showing levels below the lower limit of quantification. These data demonstrate that anti-BCMA×anti-CD3 treatment is sufficient to significantly reduce serum HDM-specific IgE 2 weeks after administration.

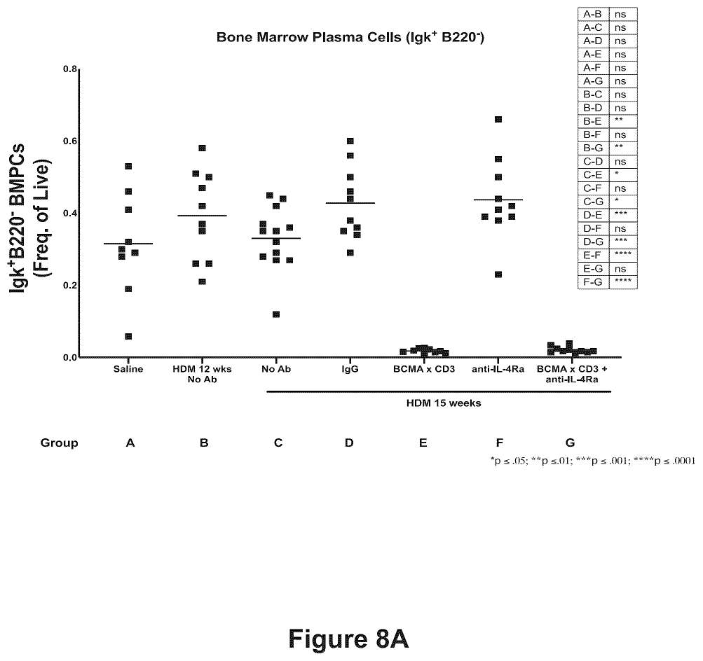

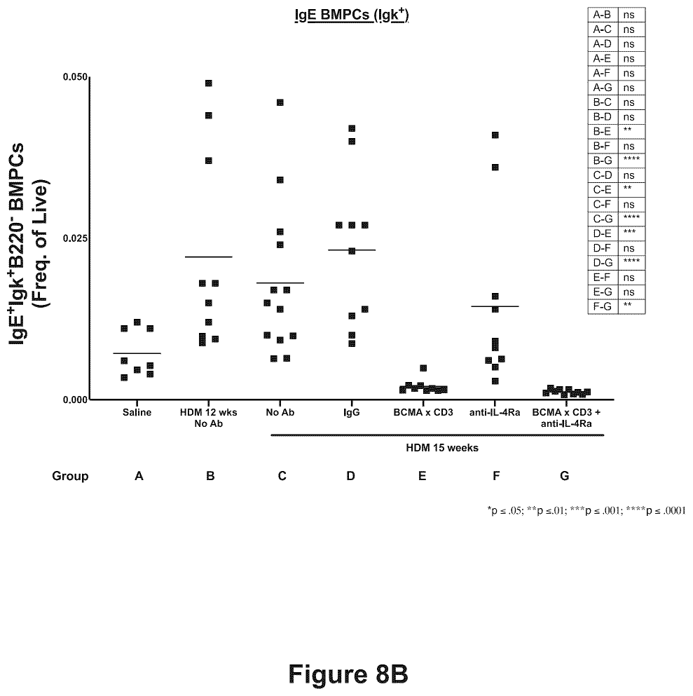

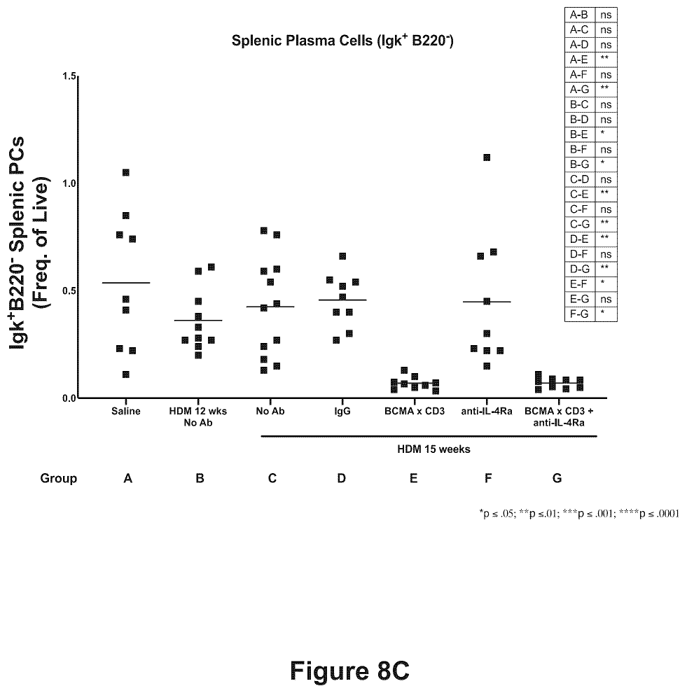

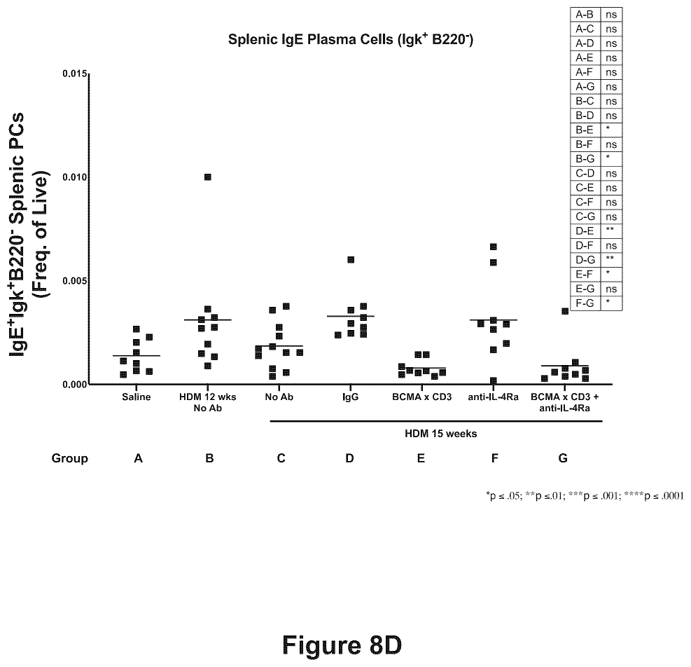

In the same experiment, anti-IL4Ra treatment did not impact total bone marrow plasma cells (FIG. 8A) and showed a trend towards reduced IgE bone marrow plasma cells (FIG. 8B). Anti-BCMA×anti-CD3 treatment alone led to a significant reduction in both total and IgE bone marrow plasma cells relative to both the untreated and the isotype control treated groups at the time of harvest (two weeks after REGN5459 administration) (FIG. 8A-8B). The combination of anti-IL-4Rα with anti-BCMA×anti-CD3 treatment also significantly reduced total and IgE-specific bone marrow plasma cells to a similar extent as the anti-BCMA×anti-CD3 alone, demonstrating that the latter treatment is sufficient to deplete bone marrow plasma cells. Similar results were also observed in the spleen; anti-IL4Ra treatment showed no impact on total or IgE splenic plasma cells (FIG. 8-8N). Anti-BMA×anti-CD3 treatment alone led to a significant reduction in total splenic plasma cells relative to both the untreated and the isotype control treated groups at the time of harvest (two weeks after REGN5459 administration) (FIG. 8C-80). The combination of anti-IL-4Rα with anti-BCMA×ant-CD3 treatment also significantly reduced total splenic plasma cells to a similar extent as the anti-BCMA×anti-CD3 alone, demonstrating that the latter treatment is sufficient to deplete splenic plasma cells. IgE plasma cells were reduced both with anti-B3MA×anti-CD3 treatment alone as well as with the combination of anti-IL-4Rα and anti-BCMA×anti-CD3. However, the reduction in IgE splenic plasma cells only achieved statistical significance when compared to the group that received HDM for 12 weeks in the absence of antibody treatment or to the isotype control group, but not when compared to the group that received HDM for 15 weeks and no antibody treatment.

TABLE 7
Effect of anti-BCMA × anti-CD3 bispecific antibody, alone or in
combination with anti-IL-4Rα antibody on serum HDM-specific IgE levels

| | | B | | | | | G |
|---|---|---|---|---|---|---|---|
| | A | (HDM 12 weeks) | C | D | E | F | (BCMA × CD3 + |
| Group | (Saline) | No Ab | (No Ab) | (IgG) | (BCMA × CD3) | (Anti-IL-4Rα) | anti-IL-4Rα) |
| Mean ± SD | 0.223 ± 0.669 | 55.37 ± 34.3 | 112.28 ± 108.88 | 139.13 ± 48.02 | 0.79 ± 2.51 | 31.25 ± 32.73 | 0.06 ± 0.13 |
| serum IgE | | | | | | | |
| (ng/mL) | | | | | | | |
| Mean rank | −47.02 | −13.5 | −9.185 | N/A | −46.9 | −21.4 | −46.1 |
| difference | (****) | (ns) | (ns) | | (****) | (ns) | (****) |
| compared | | | | | | | |
| to Isotype | | | | | | | |
| control | | | | | | | |

TABLE 8
Effect of anti-BMCA × anti-CD3 bispecific antibody,
alone or in combination with anti-IL-4Rα antibody,
on total and IgE bone marrow and splenic plasma cells

| | | | Mean Percent | |
|---|---|---|---|---|
| | | | Decrease Relative | |
| | | | to Average | |
| | | Plasma cells | Plasma Cells in | |
| | | (percent of | isotype control | |
| | Treatment | Live ± SD) | group | |
| Total BMPCs | Anti-BCMA × | 0.018 ± 0.006 | 95.76 | (***) |
| | Anti CD3 | | | |
| | Anti-IL4Ra | 0.437 ± 0.115 | −2.10 | (ns) |
| | Anti-BCMA × | 0.021 ± 0.009 | 94.83 | (***) |
| | Anti CD3 + | | | |
| | Anti-IL4Ra | | | |
| IgE BMPCs | Anti-BCMA × | 0.002 ± 0.001 | 91.08 | (***) |
| | Anti CD3 | | | |
| | Anti-IL4Ra | 0.014 ± 0.013 | 37.62 | (ns) |
| | Anti-BCMA × | 0.001 ± 0.0003 | 94.65 | (****) |
| | Anti CD3 + | | | |
| | Anti-IL4Ra | | | |
| Total | Anti-BCMA × | 0.07 ± 0.03 | 84.67 | (**) |
| Splenic PCs | Anti CD3 | | | |
| | Anti-IL4Ra | 0.448 ± 0.319 | 11.75 | (ns) |
| | Anti-BCMA × | 0.07 ± 0.024 | 86.64 | (**) |
| | Anti CD3 + | | | |
| | Anti-IL4Ra | | | |
| IgE Splenic | Anti-BCMA × | 0.00079 ± 0.0004 | 75.98 | (**) |
| PCs | Anti CD3 | | | |
| | Anti-IL4Ra | 0.0031 ± 0.002 | 14.97 | (ns) |
| | Anti-BCMA × | 0.0009 ± 0.001 | 73.81 | (**) |
| | Anti CD3 + | | | |
| | Anti-IL4Ra | | | |

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

What is claimed is:

1. A method of reducing or eliminating allergen-specific serum IgE in a subject, comprising: (a) selecting a subject with an allergic disease or disorder, a mast cell activation disorder or mastocytosis; and (b) administering to the subject in need thereof a therapeutically effective amount of an IL-4/IL-13 pathway inhibitor and a plasma cell ablating agent.

2. A method of treating allergy or preventing or reducing the severity of an allergic reaction to an allergen, comprising: (a) selecting a subject with an allergic disease or disorder, a mast cell activation disorder, or mastocytosis; and (b) administering to the subject in need thereof a therapeutically effective amount of an IL-4/IL-13 pathway inhibitor and a plasma cell ablating agent.

3. A method for treating allergy or preventing or reducing the severity of an allergic reaction to an allergen, comprising: (a) selecting a subject with an allergic disease or disorder, a mast cell activation disorder, or mastocytosis, wherein the subject is on a background therapy regimen comprising one or more doses of an IL-4/IL-13 pathway inhibitor; and (b) administering to the subject at least one dose of a plasma cell ablating agent.

4. (canceled)

5. The method of claim 1, wherein the allergic disease or disorder is selected from the group consisting of allergic asthma, hay fever, chronic urticaria, food allergy, pollen allergy, and allergy due to an environmental (non-food) allergen.

6-11. (canceled)

12. A method of increasing the efficacy and/or tolerability of an immunotherapy regimen in a subject having an allergy, the method comprising administering to the subject an IL-4/IL-13 pathway inhibitor and a plasma cell ablating agent prior to or concurrent with the immunotherapy regimen.

13. The method of claim 12, wherein the immunotherapy is oral immunotherapy.

14. The method of claim 12, wherein the immunotherapy is subcutaneous immunotherapy.

15. The method of claim 12, wherein the subject has a food allergy.

16. The method of claim 15, wherein the subject has an allergy to milk, a dairy product, egg, celery, sesame, wheat, meat, fruit, soy, fish, shellfish, a sugar, peanut, a legume, or a tree nut.

17. The method of claim 12, wherein the subject has an allergy to a non-food allergen selected from the group consisting of dust, dust mite, pollen, insect venom, mold, animal fur, animal dander, wool, latex, a metal, a household cleaner, a detergent, cosmetics, perfume, a drug, a therapeutic monoclonal antibody, ragweed, grass and birch.

18. The method of claim 12, wherein the plasma cell ablating agent is administered prior to the onset of the immunotherapy regimen.

19. The method of claim 12, wherein at least one dose of the IL-4/IL-13 pathway inhibitor is administered prior to the onset of the immunotherapy regimen.

20. The method of claim 12, wherein the IL-4/IL-13 pathway inhibitor is administered concurrently with the immunotherapy regimen.

21. The method of claim 12, wherein the IL-4/IL-13 pathway inhibitor is selected from the group consisting of an anti-IL-4 antibody, an anti-IL-13 antibody, an anti-IL-4/IL-13 bispecific antibody, an IL-4 receptor (IL-4R) inhibitor, an IL-4 trap, an IL-13 trap, and an anti-IL-4R antibody.

22. The method of claim 12, wherein the IL-4/IL-13 pathway inhibitor is an anti-IL-4 antibody.

23. The method of claim 12, wherein the IL-4/IL-13 pathway inhibitor is an anti-IL-13 antibody.

224. The method of claim 12, wherein the IL-4/IL-13 pathway inhibitor is an anti-IL-4/IL-13 bispecific antibody.

25. The method of claim 12, wherein the IL-4/IL-13 pathway inhibitor is an IL-4R inhibitor.

26. The method of claim 12, wherein the IL-4/IL-13 pathway inhibitor is an anti-IL-4R antibody.

27. The method of claim 26, wherein the anti-IL-4R antibody comprises three heavy chain complementarity determining regions (CDRs) (HCDR1, HCDR2 and HCDR3) and three light chain CDRs (LCDR1, LCDR2 and LCDR3), wherein HCDR1 has the amino acid sequence of SEQ ID NO: 3, HCDR2 has the amino acid sequence of SEQ ID NO: 4, HCDR3 has the amino acid sequence of SEQ ID NO: 5, LCDR1 has the amino acid sequence of SEQ ID NO: 6, LCDR2 has the amino acid sequence of SEQ ID NO: 7, and LCDR3 has the amino acid sequence of SEQ ID NO: 8.

28. The method of claim 27, wherein the anti-IL-4R antibody comprises a heavy chain variable region (HCVR) comprising the amino acid sequence of SEQ ID NO: 1 and a light chain variable region (LCVR) comprising the amino acid sequence of SEQ ID NO: 2.

29. The method of claim 26, wherein the anti-IL-4R antibody comprises a heavy chain and a light chain, wherein the heavy chain has an amino acid sequence of SEQ ID NO: 9.

30. The method of claim 26, wherein the anti-IL-4R antibody comprises a heavy chain and a light chain, wherein the light chain has an amino acid sequence of SEQ ID NO: 10.

31. The method of claim 26, wherein the anti-IL-4R antibody comprises a heavy chain and a light chain, wherein the heavy chain has an amino acid sequence of SEQ ID NO: 9 and the light chain has an amino acid sequence of SEQ ID NO: 10.

32. The method of claim 12, wherein the IL-4/IL-13 pathway inhibitor is dupilumab or a bioequivalent thereof.

33. The method of claim 12, wherein the IL-4/IL-13 pathway inhibitor is selected from the group consisting of dupilumab, pascolizumab, AMG317, MEDI2045, MEDI9314, tralokinumab, lebrikzimab, anrukinzumab, dectrekumab, GSK679586, MEDI7836, romilkimab, an IL-4 trap, an IL-13 trap, AER-003, and pitrakinra.

34. The method of claim 12, wherein the plasma cell ablating agent is selected from the group consisting of a B-cell maturation antigen (BCMA) targeting agent, a proteasome inhibitor, a histone deacetylase inhibitor, a BAFF inhibitor, and an APRIL inhibitor.

35. The method of claim 34, wherein the BCMA targeting agent is selected from the group consisting of an anti-BCMA/anti-CD3 bispecific antibody, a chimeric antigen receptor against BCMA (“BCMA CAR”), and an anti-BCMA antibody conjugated to a cytotoxic agent (“BCMA ADC”).

36. The method of claim 12, wherein the plasma cell ablating agent is an anti-BCMA/anti-CD3 bispecific antibody or antigen-binding fragment thereof comprising (a) a first antigen-binding domain that specifically binds to BCMA; and (b) a second antigen-binding domain that specifically binds CD3.

37. The method of claim 36, wherein the first antigen-binding domain comprises three heavy chain CDRs (HCDR1, HCDR2 and HCDR3) contained with a heavy chain variable region (HCVR) comprising the amino acid sequence of SEQ ID NO: 12, and three light chain CDRs (LCDR1, LCDR2 and LCDR3) contained with a light chain variable region (LCVR) comprising the amino acid sequence of SEQ ID NO: 20.

38. The method of claim 37, wherein HCDR1 has the amino acid sequence of SEQ ID NO: 14, HCDR2 has the amino acid sequence of SEQ ID NO: 16, HCDR3 has the amino acid sequence of SEQ ID NO: 18, LCDR1 has the amino acid sequence of SEQ ID NO: 22, LCDR2 has the amino acid sequence of SEQ ID NO: 24, and LCDR3 has the amino acid sequence of SEQ ID NO: 26.

39. The method of claim 36, wherein the second antigen-binding domain comprises three heavy chain CDRs (HCDR1, HCDR2 and HCDR3) contained with a heavy chain variable region (HCVR) comprising the amino acid sequence selected from the group consisting of SEQ ID NOs: 28 and 36, and three light chain CDRs (LCDR1, LCDR2 and LCDR3) contained with a light chain variable region (LCVR) comprising the amino acid sequence of SEQ ID NO: 20.

40. The method of claim 39, wherein HCDR1 has the amino acid sequence of SEQ ID NO: 30 or 38, HCDR2 has the amino acid sequence of SEQ ID NO: 32 or 40, HCDR3 has the amino acid sequence of SEQ ID NO: 34 or 42, LCDR1 has the amino acid sequence of SEQ ID NO: 22, LCDR2 has the amino acid sequence of SEQ ID NO: 24, and LCDR3 has the amino acid sequence of SEQ ID NO: 26.

41. The method of claim 36, wherein the anti-BCMA/anti-CD3 bispecific antibody or antigen-binding fragment thereof comprises:
(a) a first antigen-binding domain that comprises HCDR1, HCDR2, and HCDR3 domains, respectively, comprising the amino acid sequences of SEQ ID NOs: 14, 16, and 18, and LCDR1, LCDR2, and LCDR3 domains, respectively, comprising the amino acid sequences of SEQ ID NOs: 22, 24, and 26; and
(b) a second antigen binding domain that comprises HCDR1, HCDR2, and HCDR3 domains, respectively, comprising the amino acid sequences of SEQ ID NOs: 30, 32, and 34, and LCDR1, LCDR2, and LCDR3 domains, respectively, comprising the amino acid sequences of SEQ ID NOs: 22, 24, and 26.

42. The method of claim 36, wherein the anti-BCMA/anti-CD3 bispecific antibody or antigen-binding fragment thereof comprises:
(a) a first antigen-binding domain that comprises HCDR1, HCDR2, and HCDR3 domains, respectively, comprising the amino acid sequences of SEQ ID NOs: 14, 16, and 18, and LCDR1, LCDR2, and LCDR3 domains, respectively, comprising the amino acid sequences of SEQ ID NOs: 22, 24, and 26; and
(b) a second antigen binding domain that comprises HCDR1, HCDR2, and HCDR3 domains, respectively, comprising the amino acid sequences of SEQ ID NOs: 38, 40, and 42, and LCDR1, LCDR2, and LCDR3 domains, respectively, comprising the amino acid sequences of SEQ ID NOs: 22, 24, and 26.

43. The method of claim 12, wherein the IL-4/IL-13 pathway inhibitor is administered prior to the plasma cell ablating agent.

44. The method of claim 12, wherein the IL-4/IL-13 pathway inhibitor is administered after the plasma cell ablating agent.

45. The method of claim 12, wherein the IL-4/IL-13 pathway inhibitor and the plasma cell ablating agent are administered concurrently.

46-48. (canceled)
